(19) United States
(12) Patent Application Publication
(10) Pub. No.: US 2021/0254053 A1
(43) Pub. Date: Aug. 19, 2021

(54) METHODS AND USES OF HIGH-THROUGHPUT INFERENCE OF SYNAPTIC CONNECTIVITY RELATIONSHIPS AMONG CELL TYPES

(71) Applicant: PRESIDENT AND FELLOWS OF HARVARD COLLEGE, Cambridge, MA (US)

(72) Inventors: Steve MCCARROLL, Cambridge, MA (US); Arpiar SAUNDERS, Cambridge, MA (US)

(21) Appl. No.: 17/246,200

(22) Filed: Apr. 30, 2021

Related U.S. Application Data
(63) Continuation of application No. PCT/US2019/059205, filed on Oct. 31, 2019.
(60) Provisional application No. 62/755,052, filed on Nov. 2, 2018.

(51) Int. Cl. C12N 15/10; C12N 15/86

(52) U.S. Cl. CPC C12N 15/1093; C12N 2760/20143; C12N 2760/20131; C12N 15/86

(57) ABSTRACT

Embodiments of the disclosure are directed to a viral genome, such as for example, a rabies virus (RV) genome, a viral particle comprising a viral genome, a polynucleotide encoding barcode, a method of constructing a hyper-diverse barcoded plasmid library, a library of hyper-diverse barcoded plasmids, and a method of inferring synaptic connectivity from identifiable viral barcodes and identifying cell types or cell type information, systems, and uses of identifying each cell's RV particles in the course of sequencing its RNAs, including the identification of sets of cells that are within the same synaptic network, while simultaneously or sequentially ascertaining the molecular identity and state of each cell, for example, from its pattern of RNA expression.

### CROSS REFERENCE TO RELATED APPLICATIONS

The present application is the US national stage and a continuation of International Application No. PCT/US2019/059205, filed Oct. 31, 2019, which claims the benefit of and priority to U.S. Provisional Application No. 62/755,052, filed Nov. 2, 2018, each of which is incorporated herein by reference in its entirety.

### FIELD OF THE DISCLOSURE

The disclosure generally relates to the field of synaptic connectivity relationships. In particular, the disclosure provides methods and uses for quickly and inexpensively mapping synaptic connectivity relationships among a large number of brain cells through high-throughput DNA sequencing.

### BACKGROUND

The brain is made of billions of individual cells, of many functionally distinct cell types, which are wired together through synapses into synaptic networks. The architecture or wiring diagram of these networks, including the quantitative connectivity relationships among different cell types within the same networks, determines how information is processed by neural networks. At the scale of the whole brain, these networks are called the “connectome.”

Connectomes are highly dynamic on evolutionary time-scales and over the course of an individual's life. Patterns of connectivity evolved to support behavioral specialization across species over the course of thousands and even millions of years. Over the course of an individual's life, synaptic networks undergo genetically programmed changes, which are particularly prevalent during embryonic and post-natal development. Changes in synaptic networks also reflect and in fact underpin an individual's unique experience, supporting learning and habit formation, both normal and self-destructive. Connectivity changes are also a major component of the pathological and/or compensatory response of the brain to neurodegenerative and neuropsychiatric conditions and probably represent a key point of disease intervention.

Measurements of synaptic connections are typically made through electrophysiological recordings or through anatomical reconstructions using electron-microscopy (EM). These techniques have intrinsic limitations that prevent systematic, scalable, and cell-type-informed inference of synaptic networks. For example, EM reconstructions are limited to small volumes of less than 0.125 mm³ and typically exclude the molecular identification of cells, while the known high-throughput reconstruction experiments take many months to acquire and analyze anatomical data. Electrophysiological and anatomical reconstructions of synaptic networks are also restricted to tens or hundreds of cells. Moreover, many published studies are based on single datasets, and are thus not amenable to statistical analysis of connectivity across multiple subjects let alone experimental conditions. Electrophysiological measurements of synaptic connectivity have traditionally been limited to neighboring cells in acute slices with molecular identifiers. Transgenic mice in combination with optogenetic activation have allowed genetically-defined cell types to be activated or recorded from, thus improving the throughput of cell-type informed connectivity measurements. However, even with these improvements, electrophysiological experiments have many limitations. For example, experiments are limited to transgenic animals, which need to be engineered and interbred for every cell type pair, which is a prohibitively long and expensive process, even for a single region.

Other systems of inferring synaptic connectivity through DNA sequencing have been proposed which involve, for example, creating a unique genomic locus in each cell using a recombinase system similar to “brainbow” where exogenous recombinases are applied to generate diverse combinations of colors for cellular tagging. However, this method failed to have a known sequencing technology that could read out barcodes and how the shuffled genomic barcodes were related to the synaptic connections or cell type identity. Another system of inferring synaptic connectivity called “MAPseq” based on barcode carrying sindbis-viruses which allow the quantification of projection anatomy of a small number of single cells through sequencing was also proposed. The sindbis virus does not transit synapses. To extend their sindbis system to inform synaptic connections, they proposed to use multiple viral injections in different brain regions. To identify synaptic connections, the infected cells would enrich mRNA barcodes in the pre- and post-synaptic compartments where mRNA barcodes could biochemically fuse through spatial proximity (called “SYNseq”) and then the fused mRNAs readout through sequencing. In practice, the fusion process was almost completely inefficient. As an alternative readout for synapse spanning barcodes, in situ sequencing techniques are being developed, but no studies have yet been published demonstrating successful sequencing of synapse-localized barcodes nor it is clear how the molecular identity of the barcoded cells could be established.

In spite of the foundational importance of synaptic connectivity for all of brain biology, systematically ascertaining the multitude of synaptic connections (and thereby measuring key parameters of connectomes) even from a single region of a single mammalian species on a single genetic background is currently intractable. The difficulty is due to the small size (<0.5 mm), large number (>1000 per cell), and dense packing of synapses and is exacerbated by the often-long distances that separate cells within the same network. Accordingly, there is a need for a rapid and economic high-throughput means for ascertaining the synaptic connectivity relationships among the billions of brain cells.

### SUMMARY OF THE INVENTION

As described below, an aspect of the disclosure is the inference of cell-type specific synaptic networks coupled with sequencing, aided by the synapse-specific spread of a virus (e.g., Rabies Virus (RV)). Another aspect of the disclosure involves using molecular-biological and virological methods to generate DNA plasmids with hyper diverse-barcodes and then packaging such libraries, for example, in viral particles (e.g., RV particles) that maintain hyper-diverse barcodes in the genomes. A further aspect of the disclosure provides plasmids and viruses that were generated to perform an immense variety of connectivity-tracing experiments, as well as identify cell types.

In one aspect, embodiments of the disclosure are directed to a rabies virus genome, comprising: a 3′ to 5′ linear, nucleic acid sequence encoding a rabies virus (RV) nucleoprotein, a RV phosphoprotein, a RV matrix protein, a barcode, and a RV polymerase. Another aspect of the rabies virus genome may be directed to, the barcode gene which may comprise a restriction enzyme cassette. In another aspect, one or more genes encoding the RV nucleoprotein, the RV phosphoprotein, the RV matrix protein, the barcode, or the RV polymerase, may be an endogenous gene or a transgene. A further aspect may be directed to a selectable moiety, selectable marker, or detectable moiety, including but not limited to a fluorophore, where the fluorophore is a fluorescent protein, such as but not limited to, a green, red, or yellow fluorescent protein, for example, an enhanced green fluorescent protein. Yet another aspect may be directed to the rabies virus genome comprising a restriction enzyme cassette that divides the barcode into two halves.

Another aspect may be directed to a viral genome, comprising: a nucleic acid sequence encoding viral proteins of a viral species and a barcode, wherein said viral genome is in a viral particle of the viral species that infects through synaptic junctions. In yet another aspect, non-limiting viral species may include a rabies virus and vesicular stomatitis virus. A further aspect may be directed to a viral genome comprising a nucleic acid sequence encoding all viral proteins of the viral species. The viral genome of yet another aspect may be directed to the barcode comprising a restriction enzyme cassette. In yet a further aspect, the viral genome may further comprise a selectable marker or detectable moiety, where the detectable moiety is a fluorophore, such as for example, a green, red, or yellow fluorescent protein, including but not limited to an enhanced green fluorescent protein.

In one aspect, a viral particle comprises the viral genome described herein. Another aspect may be directed to a rabies virus particle comprising the rabies virus genome described here.

In another aspect, embodiments of the disclosure are directed to a polynucleotide encoding a barcode, comprising a restriction enzyme cassette, where the restriction enzyme cassette separates the barcode into two equal or unequal halves. A further aspect may be directed to a selectable marker, detectable moiety, or selectable moiety, including but not limited to a fluorophore, where the fluorophore is a fluorescent protein, such as an enhanced green fluorescent protein.

Another aspect of embodiments of the disclosure is directed to a method of constructing a hyper-diverse barcoded plasmid library, comprising:
a) amplifying a template plasmid in an amplification reaction using a forward primer and a reverse primer, wherein the forward primer and the reverse primer each comprise from 5′ to 3′ a complementary region, a barcode, which in some aspects, may comprise a degenerate sequence or a semi-degenerate sequence, a linker, and a restriction enzyme site, where the restriction enzyme sites of the forward primer and the reverse primer generate 3′ compatible overhangs when cleaved;
b) generating double-stranded linear amplicons, each comprising the template plasmid sequence comprising barcodes at the termini of the amplicons;
c) digesting the amplicon with a restriction enzyme to produce a digested product comprising 5′ and 3′ overhangs;
d) ligating the digested product to produce a circular bipartite-barcoded plasmid; and
e) selectively digesting linear DNA with an exonuclease.
   Another aspect of embodiments of the disclosure is directed to RecBCD as the exonuclease. A further aspect may be directed to the barcode sequence (of the forward primer or the reverse primer), where the barcode sequence may be a degenerate or semi-degenerate sequence, and the barcode sequence may be separated from the compatible restriction enzyme site by 3 base pairs to 25 base pairs, or 3 base pairs to 5 base pairs, or 5 base pairs. Another aspect may be directed to the linker sequence (of the forward primer or the reverse primer) of 3 base pairs to 30 base pairs in length, or 3 base pairs to 5 base pairs in length, or 5 base pairs in length. A further aspect may be directed to the complementary region (of the forward primer or the reverse primer), where the complementary region is homologous to the template plasmid, being 18 base pairs to 200 base pairs in length or 54 base pairs in length. Another aspect may be directed to the barcode sequence (of the forward primer or the reverse primer) of 3 base pairs to 30 base pairs in length, or 3 base pairs to 10 base pairs in length, or 10 base pairs in length. In one embodiment, the barcode sequence is a degenerate barcode sequence, and in another embodiment, the barcode sequence is a semi-degenerate sequence.

In one aspect, embodiments of the disclosure may be directed to a library of hyper-diverse barcoded plasmids, wherein the hyper-diverse barcoded plasmid is a circular plasmid and comprises at least two identifiable barcode sequences separated from a restriction enzyme site by a linker sequence. A further aspect may be directed to the hyper-diverse barcoded plasmid that is a circular plasmid and comprises three identifiable barcode sequences, four identifiable barcode sequences, five identifiable barcode sequences, six identifiable barcode sequences, or more. In yet another aspect, the barcode is highly variable across the individual viral genomes or particles in a library. A further aspect may be directed to a degenerate sequence or a semi-degenerate sequence. Another aspect may be directed to a library of hyper-diverse barcoded plasmids, where the hyper-diverse barcoded plasmids are constructed by the method of constructing a hyper-diverse barcoded plasmid library described here.

A further aspect may be directed to a method of inferring synaptic connectivity, comprising:
a) contacting one or more starter cells susceptible to infection with a library of viral particles each comprising a rabies virus genome described herein, where the RV genome comprises: a 3′ to 5′ linear, nucleic acid sequence encoding a rabies virus (RV) nucleoprotein, a RV phosphoprotein, a RV matrix protein, a barcode, and a RV polymerase, with or without a selectable marker or detectable moiety, where the barcode comprises a restriction enzyme cassette, or where the barcode is identifiable;
b) replicating the virus particle within the one or more infected starter cells;
c) allowing the virus particle to infect one or more additional cells, each synaptically connected to the starter cell;
d) optionally sorting cells to select the rabies virus infected cells;
e) creating single-cell RNA-seq libraries from the resulting or infected cells;
f) high-throughput sequencing of the single-cell RNA-seq libraries to identify RNA sequences and viral barcodes present within each cell; and
g) inferring synaptic connectivity of cells based on the sharing of at least one identifiable barcode, wherein cells that share an identifiable barcode are inferred to be in the same synaptic network.

In one aspect, the invention provides a rabies virus genome containing a 3′ to 5′ linear, nucleic acid sequence encoding a rabies virus (RV) nucleoprotein, a RV phosphoprotein, a RV matrix protein, a barcode, and a RV polymerase. In one embodiment, the one or more nucleic acid sequences encoding the RV nucleoprotein, the RV phosphoprotein, the RV matrix protein, the barcode, or the RV polymerase is an endogenous gene or transgene.

In another aspect, the invention provides a viral genome containing a nucleic acid sequence encoding some viral proteins of a viral species and a barcode, where the viral genome is in a viral particle of the viral species that infects through synaptic junctions. In one embodiment, the nucleic acid sequence encodes all viral proteins of the viral species.

In another aspect, the invention provides a rabies virus particle containing the rabies virus genome of any previous aspect.

In another aspect, the invention provides a viral particle containing the viral genome of any previous aspect or any other aspect of the invention delineated herein.

In another aspect, the invention provides a polynucleotide encoding a barcode containing a restriction enzyme cassette, where the restriction enzyme cassette separates the barcode into two equal or unequal halves.

In another aspect, the invention provides a method of constructing a hyper-diverse barcoded plasmid library, involving
a) amplifying a template plasmid in an amplification reaction using a forward primer and a reverse primer, where the forward primer and the reverse primer each comprise from 5′ to 3′ a complementary region, a barcode, a linker, and a restriction enzyme site, where the restriction enzyme sites of the forward primer and the reverse primer generate 3′ compatible overhangs when cleaved;
b) generating double-stranded linear amplicons, each containing the template plasmid sequence containing barcodes at the termini of the amplicons;
c) digesting the amplicon with a restriction enzyme to produce a digested product containing 5′ and 3′ overhangs;
d) ligating the digested product to produce a circular barcoded plasmid; and
e) selectively digesting linear DNA with an exonuclease. In one embodiment, the exonuclease is RecBCD.
   In one embodiment, the barcode sequence is separated from the restriction enzyme site by 3 base pairs to 25 base pairs (e.g., 3, 5, 10, 15, 20, 21, 22, 23, 24, 24, 25). In another embodiment, the linker sequence is 3 base pairs to 30 base pairs (e.g., 3, 5, 10, 15, 20, 25, 26, 27, 28, 29, 30) in length. In another embodiment, the complementary region is 18 base pairs to 200 base pairs (e.g., 18, 19, 20, 30, 40, 50, 60, 70, 80, 90, 100, 125, 150, 175, 200) in length.

In another aspect, the invention provides a library of hyper-diverse barcoded plasmids, where the hyper-diverse barcoded plasmid is a circular plasmid and contains at least two identifiable barcode sequences separated from a restriction enzyme site by a linker sequence. In one embodiment, the hyper-diverse barcoded plasmid is constructed by the method of a previous aspect.

In another aspect, the invention provides a method of inferring synaptic connectivity, involving:
a) contacting one or more starter cells susceptible to infection with a library of viral particles each containing a rabies virus genome of a previous aspec;
b) replicating the virus particle within the one or more infected starter cells;
c) allowing the virus to infect one or more additional cells, each synaptically connected to the starter cell;
d) optionally sorting the cells to select the rabies virus infected cells;
e) creating single-cell RNA-seq libraries from the infected cells;
f) high-throughput sequencing of the single-cell RNA-seq libraries to identify RNA sequences and viral barcodes present within each cell; and
g) inferring synaptic connectivity of cells based on the sharing of at least one identifiable barcode, where cells that share an identifiable barcode are inferred to be in the same synaptic network.
   In one embodiment, the method further involves identifying cell types from the identified RNA sequences. In another embodiment, the identifiable barcode contains a selectable marker or detectable moiety. In another embodiment, the selectable marker or detectable moiety is a fluorophore, an antibody resistance cassette, a capture molecule, a biotin molecule, streptavidin molecule, or an antigen.

In various embodiments of any of the above aspects or any other aspect of the invention delineated herein, the barcode contains a restriction enzyme cassette. In various embodiments of any of the above aspects or any other aspect of the invention delineated herein, the rabies virus genome contains a selectable marker or detectable moiety, such as a fluorophore. In one embodiment, the fluorophore is a fluorescent protein. In another embodiment, the fluorophore is green, red, or yellow fluorescent protein. In another embodiment, the restriction enzyme cassette divides the barcode into two halves. In another aspect, the method of inferring synaptic connectivity may further comprise identifying cell types or cell type information from the identified RNA sequences. Another aspect may be directed to a method of simultaneously or sequentially, in any order, inferring synaptic connectivity and identifying cell types or cell type information.

In various embodiments of the above aspects, the identifiable barcode comprises a selectable marker or detectable moiety, which includes but is not limited to, a fluorophore, an antibody resistance cassette, a capture molecule, a biotin molecule, streptavidin molecule, or an antigen. In another aspect, the selectable marker or detectable moiety of the rabies virus genome may include, and is not limited to, a fluorophore, an antibody resistance cassette, a capture molecule, a biotin molecule, streptavidin molecule, or an antigen. In other embodiments of the above aspects, the virus is a rabies virus or vesicular stomatitis virus.

### DESCRIPTION OF THE FIGURES

The characteristics and advantages of embodiments of the disclosure will be described in detail in conjunction with the accompanying figures.

FIGS. 1A-1I show the generation of Rabies Virus (RV) libraries carrying hyper-diverse genomic barcodes. FIG. 1A shows a schematic of the SBARRO (Synaptic barcode analysis with a retrograde rabies readout) concept for sequencing-based inference of synaptic connectivity among molecularly-defined cell types. A library of EnvA-pseudotyped and G-deleted RV or RVdG particles may be used to transduce TVA⁺ “starter” cells. In “starter” cells, RV particles replicate and undergo monosynaptic retrograde spread (due to the Glycoprotein “G” gene) thus expressing barcoded enhanced Green Fluorescent Protein (EGFP) mRNA in the pre-synaptic network. Cells sharing viral barcodes (VBCs) are part of the same synaptic network. “Starter” and “presynaptic” cells are distinguished transcriptionally. FIG. 1B shows a schematic of the barcode region of the RV genome comprising, linearly from 3′ to 5′, genes which encode the nucleoprotein (N), phosphoprotein (P), matrix protein (M), enhanced green fluorescent protein (eGFP) which replaces the glycoprotein (G) gene, and polymerase (L). Two 10 basepair (bp) barcodes separated by restriction cassette are introduced between the 3′ end of EGFP and the polyadenylation sequence. The combined 20 bp barcode can distinguish more than about 10¹² distinct sequences. FIG. 1C shows the workflow for packaging hyper-diverse barcoded RV: circular plasmid, supercoiled plasmid, RV, and pseudo-typed RV. First, barcoded circular DNA plasmids encoding the RV genome were generated with a novel bi-partite barcoding procedure (see, FIG. 3A). Circular barcoded plasmids were sufficiently abundant (>2 μg) and diverse (see below) to approach theoretical limits of all possible 20 bp combinations. Second, to generate super-coiled plasmids while maintaining barcode diversity, DNA was extracted from transfected E. coli grown on large plates, which preserved barcode diversity better than liquid culture (see, FIG. 3D). Third, super-coiled barcoded plasmids were rescued into infective RV particles followed by Env-based pseudotyping through a novel, transfection-based packaging protocol that reduced barcode loss and skew by minimizing RV amplification steps (see, FIG. 3E). FIG. 1D shows the number of unique barcodes (10-25 million sequence reads) from each step of RV packaging. DNA plasmid, RV RNA genome, and RV EGFP transcript barcodes were counted with UMI-based systems (see, FIG. 3B) and raw barcode sequence data was corrected for library-preparation or sequencing-based mutations (see, FIG. 3C) which otherwise inflate barcode diversity proportionally to the depth of sequencing. FIG. 1E shows the cumulative distribution of sampled barcode diversity by “Abundance Group (AG)” and an exploded view restricted to AG 1-25 (Right) (at 25, from Top red line to Bottom blue line: Circular plasmid, orange Supercoiled plasmid, green RV G, and RV EnvA). AGs bin barcodes sharing the same number of sequencing counts (e.g., AG=1 contains barcodes that were only counted once; AG=2 contains barcodes counted twice). FIG. 1F shows the distribution of “Founder Viral Barcode (FVB)” thresholds for viral barcodes (VBCs) in the pseudotyped (lower line) and non-pseudotyped (upper line) RV libraries. FVB thresholds are calculated as the maximum number of samples drawn from each library before the probability of re-drawing the same VBC>1%. FVB thresholds help determine how much of the barcoded library may be trustworthy in seeding single infections for an experiment of a given size. Only the FVB thresholds of less than 1000 are shown. The diversity metrics based on synthetic combinations of distinct barcoded packagings (n=1, 2, 5 or 10) illustrate that SBARRO is scalable to hundreds of thousands or millions of cells (FIGS. 1G-I). FIG. 1G shows unique sampled barcodes by the number of RV packagings, cumulative distribution by AG (FIG. 1H) by the number of RV packagings with exploded view restricted to AB 1-25 (Right) (Number of RV Packagings for Top purple line to Bottom green line: 10, blue line: 5, yellow line: 2, and 1), and FVB threshold distributions by the thousands (K) are shown. FIG. 1I shows the FVB threshold distributions (at 5, Number of RV Packagings for Top purple line to Bottom green line: 10, blue line: 5, yellow line: 2, and 1).

FIG. 2 shows a schematic for barcoding the rabies virus cDNA with two unique barcodes of 10 base pairs each to produce circular barcoded plasmids.

FIGS. 3A-3E show methods of creating and quantifying hyper-diverse barcodes in plasmids and RV genomes. FIG. 3A shows a PCR-based technique for generating high-quality circular plasmids carrying hyper-diverse barcodes. Left, bi-partite 10 basepair (bp) randomer barcodes introduced through forward and reverse PCR primers targeting the desired region of the non-barcoded (template) plasmid. Each primer also carries 5 bp fixed-sequence adapter and 6 bp PlutI restriction enzyme site. With each cycle of PCR, primer annealing and extension introduces novel pairs of forward and reverse barcodes on individual molecules. Completed reactions contain micrograms (˜25 μg/96 well plate) of double-stranded linear amplicons with random pairs of 10 bp barcodes on each terminal end. To modify these linear molecules into functional circular plasmids, a single tube-based system to 1) create sticky ends (PlutI digest), 2) remove remaining template (DpnI digest), 3) ligate sticky ends (T4 ligase), and 4) finally digest the remaining linear DNA (RecBCD). Barcodes to the interval between the end of the EGFP coding sequence and the RV polyadenylation site on the pSPBN-4GFP plasmid are targeted. FIG. 3B shows a double-stranded DNA (dsDNA) plasmid and RNA genome barcodes counted with Unique Molecular Identifier (UMI)-based systems. UMI-based sequence quantification occurs for barcodes in plasmids, RV-RNA genomes, and transcripts captured in 10× single-cell libraries. For double-stranded DNA (dsDNA) plasmids and RV genomes, UMI-containing oligonucleotides were hybridized and polymerase-extended through regions adjacent to the barcodes. UMI-tagged barcodes were amplified for sequencing using PCR primers containing Illumina® P5/P7 sites. To amplify VBCs from transcripts captured in 10× single-cell experiments, a P7 containing primer was targeted to the 3′ end of GFP and P5 containing primer targets the Illumina® Read 1 Primer site that serves at the PCR handle for 10× capture probes. FIG. 3C shows raw barcode sequence data corrected for library-preparation or sequencing-based mutations which otherwise inflate barcode diversity proportionally to the depth of sequencing. Barcode sequences in library (i.e., non-single cell) datasets were collapsed along a hamming edit distance 1 mutation path to account for erroneous inflation of barcodes due to mutations induced during library preparation or sequencing. To illustrate the effect of mutation path collapse (MPC), the effect of MPC on a viral barcode library of EnvA-pseudotyped barcoded B19 RV was observed. Left, total unique barcodes comparing raw to MPC. Middle, cumulative distribution of sampled barcode diversity by “Abundance Group (AG).” AGs bin barcodes sharing the same number of sequencing counts. Right, distribution of “Founder Viral Barcode (FVB)” thresholds for VBCs in the pseudotyped and non-pseudotyped RV libraries. FVB thresholds are calculated as the maximum number of samples drawn from each library before the probability of re-drawing the same VBC>1%. FIG. 3D shows super-coiled DNA barcode diversity following extraction from bacteria transformed with the same barcoded plasmid and grown on either 1) plates or 2) liquid culture. DNA transfected from E. coli grown on large plates preserved barcode diversity better than liquid culture when generating super-coiled plasmids. FIG. 3E shows RV genome barcode diversity using super-coiled DNA as input and following either 1) a previously published RV packaging protocol (Version 1) (Wickersham, et al. Nat Protoc 5:595-606, 2010) comprising the steps of Transfection (T), Amplification (A) steps, and Pseudotyping (PT) or 2) the barcode diversity optimized protocol described here comprising the steps of Transfection and Pseudotyping without any Amplification step.

FIG. 4 shows a plasmid for evaluating efficiency of the ligations and exonuclease reactions. The HEX probe crosses the ligation site while the FAM probe binds to the L RV polymerase gene, functioning as a control, where the barcode is depicted as BC.

FIGS. 5A-5F show the simultaneous single-cell measurement of endogenous transcriptomes and RV barcodes. FIG. 5A shows a schematic of the SBARRO workflow where a pseudotyped, hyper-diverse barcoded RV library infects a starter cell, which is then transduced, replicated and spread, cells may be sorted by, e.g., Fluorescence-Activated Cell Sorting (FACS) or the like, single-cell RNA sequencing (scRNA-seq) for creating a transcriptomic atlas of every cell type. FIG. 5B shows single-cell RNA profiles which identify cell class and quantify RV infection. A t-distributed Stochastic Neighbor Embedding (tSNE) plot colored by cell class for 54,260 SBARRO cells from n=7 mouse cortical cultures (Left) shows in the Cell Class panel, the majority of cells as glutamatergic neurons in the upper left quadrant, astrocytes in the lower right quadrant, oligodendrocytes in the lower right portion of the upper right quadrant, interneurons in the lower left quadrant, and polydendrocytes in the upper left portion of the upper right quadrant (see, FIG. 6A, FIG. 7C). Fraction RV transcripts (scaled to 100,000 transcripts per cell) where the rabies genome encodes five proteins, four of which are shown here: nucleoprotein (N), phosphoprotein (P), matrix protein (M), and polymerase (L), and enhanced green fluorescent protein (eGFP) (Middle), and RV fractions by cell class (Astrocyte, Glutamatergic neurons, Interneurons, Polydendrocytes, and Oligodendrocytes are identified from left to right columns; Right). FIG. 5C shows expression levels of RV genes (nucleoprotein (N), phosphoprotein (P), matrix protein (M), and polymerase (L); and enhanced green fluorescent protein (EGFP)) within glutamatergic neurons. FIG. 5D shows the correlation between viral barcode (VBC) unique molecular identifier (UMI) counts for Enhanced Green Fluorescent Protein (EGFP) and total VBC for all cells. FIG. 5E shows dendrograms depicting hamming edit distance (ED) relationships for all VBCs found within the same cell before and after within-cell barcode collapse. For this example, the VBC sequence is “CCGTGGAGTACCATCA” (SEQ ID NO: 1). Mutational processes inflate VBC diversity and counts, but can be corrected by collapsing VBCs with similar sequences within single-cells. Dendrograms depicting hamming edit distance (ED) relationships for all VBCs found within the same cell before and after within-cell barcode collapse. Random 20 bp barcodes 1) have a mean ED of 9 (upper blue dashed line) and 2) 99%>ED 2 (lower red dashed line). FIG. 5F shows the cumulative distribution of the number of unique VBCs per cell by cell class (glutamatergic neuron, interneuron, astrocyte, oligodendrocyte, polydendrocyte).

FIGS. 6A-6C show single-cell transcriptomes and RV barcodes from SBARRO experiments in mouse cortical cultures. FIG. 6A shows a tSNE plot of 54,260 cells from n=7 mouse cortical culture experiments. Cells color-coded by 1) cell class (see, FIG. 5B, FIG. 7C), 2) subcluster identities following independent component analysis (ICA)-based clustering, 3) experiment, and 4) sequencing pool (for experiments with multiple sequencing libraries). In the Cell Class panel, the largest grouping in the upper left quadrant were glutamatergic neurons, the lower left quadrant included a small grouping of interneurons, the lower right quadrant had astrocytes, while the upper right quadrant primarily included oligodendrocytes, with small portion of polydendrocytes, and even smaller portion of interneurons. In the Subcluster panel, the upper left quadrant comprised of oligodendrocytes, essentially replacing the glutamatergic neurons from the Cell Class panel. In the Experiment panel, the majority of cells are oligodendrocytes. While in the Sequencing Pool panel, the cells include interneurons, astrocytes, and polydendrocytes. FIG. 6B shows the correlation between the number of viral barcode (VBC) unique molecular identifiers (UMIs) and the number of unique VBC per cell, with (Right) and without (Left) barcode collapse. FIG. 6C shows histograms displaying hamming edit distance (ED) distributions between real VBCs in the same cell with (Right) and without (Left) collapse as compared to EDs from random 20 base pair barcodes. ED measurements were equivalently downsampled (56,063 each). Within-cell barcode collapse corrects for barcode inflation due to mutational processes.

FIGS. 7A-7D show the inference of sparse “starter” cells in mouse cortical cultures through RV barcode sharing. FIG. 7A and FIG. 7B demonstrate that transduction of sparse “starter” cells in mouse cortical with EnvA-pseudotyped barcoded RV library produces spatially clustered networks presynaptic cells. FIG. 7A shows an anatomical overview of sparse networks in cortical culture. FIG. 7B shows a magnified view of a single sparse network. FIG. 7C shows a t-distributed stochastic neighbor embedding (tSNE) projection of SBARRO single cell transcriptome data acquired from sparse network cortical culture experiments (as described in FIG. 5A; n=54,260 cells, 7 experiments/culture wells) (see, FIG. 5B, FIG. 6A). Cells in which RV transcripts carrying the example viral barcode (VBC) (Left) were captured are shown with enlarged circles, with the color of each circle corresponding to the number of VBC UMIs in each cell. The example VBC was observed in n=44 cells, the molecular identities of which were defined using their transcriptomes and mapped to different locations of the tSNE plot. To determine if any of those 44 cells were the “starter” cell, TCB expression (Right) is quantified and visualized. FIG. 7D shows SBARRO sequenced networks (Left line) contain fewer cells than anatomically-defined networks (Right line) (described in FIG. 5A) from cortical culture experiments with same numbers of starter cells. The smaller network size distribution is expected due to a loss of cells during tissue processing or a failure of cells to be captured during the creation of single-cell transcriptional libraries.

FIGS. 8A-8E provide another example dataset from which directional, cell-type-specific synaptic networks are identified using SBARRO from in vitro cultured synaptic networks derived from embryonic mice. FIG. 8A shows a cartoon schematic illustrating how tissue from developing mouse cortex and striatum is dissociated into single cells and co-cultured before SBARRO infection. FIG. 8B. shows the molecular identities of single-cell RNA profiles from SBARRO libraries. Endogenous gene expression patterns were used to annotate major cell types (dotted boxes). Of 2,607 reconstructed synaptic networks, n=113 had identifiable starter cells, a subset of which are shown in FIG. 8D. FIG. 8C plots the frequencies of cell types across all experiments. FIG. 8D plots the counts of cell types in individual presynaptic networks originating from particular starter cell types. FIG. 8E illustrates credible differences in cell-type specific networks arising from striatal spiny projection neurons (SPNs) or GABA Interneuron starter cells.

### DETAILED DESCRIPTION

Detailed embodiments of the present disclosure are disclosed herein; however, it is to be understood that the disclosed embodiments are merely illustrative of the disclosure that may be embodied in various forms. In addition, each of the examples given in connection with the various embodiments of the disclosure is intended to be illustrative, and not restrictive.

All terms used here are intended to have their ordinary meaning in the art unless otherwise provided. All concentrations are in terms of percentage by weight of the specified component relative to the entire weight of the topical composition, unless otherwise defined.

The disclosure generally features methods and uses for rapidly and economically mapping synaptic connectivity relations among the billions of brain cells. The limitations of known methods may be overcome by utilizing high-throughput DNA sequencing for inferring synaptic networks as described here. Instead of directly observing synaptic connections using electrophysiology or anatomy, synaptic connections may be inferred by tracking the infectivity paths of a large number of individual rabies virus (RV) particles (RVP) as they transit synaptic networks (in the brain, or in cells cultured in vitro) via synaptic connections. RV spreads through the nervous system exclusively through synaptic junctions. To distinguish the RV particles from one another, each individual particle's genome is given an identifying genomic sequence (“barcode”). The barcode is transcribed within infected cells; the transcript can be ascertained together or with other mRNAs, in the course of single-cell RNA analysis. The barcode sequence is “read out” through sequencing of DNA that is enzymatically derived from these RNA transcripts by reverse transcription.

Embodiments of the disclosure are directed to a virus genome (e.g., RV), a viral particle comprising a viral genome, a polynucleotide encoding barcode, a method of constructing a hyper-diverse barcoded plasmid library, a library of hyper-diverse barcoded plasmids, and a method of inferring synaptic connectivity and identifying cell types or cell type information, systems, and uses of identifying each cell's viral particles in the course of sequencing its RNAs, including the identification of sets of cells that are within the same synaptic network, while simultaneously ascertaining the molecular identity and state of each cell, for example, from its pattern of RNA expression. This allows cell-type and synaptic-network information to be ascertained simultaneously, or alternatively, sequentially in either order, i.e., identifying cell type then synaptic-network information or identifying synaptic-network information then cell type. Because RV (1) spreads in the retrograde direction (i.e., from dendrites/post-synaptic compartments into axons/pre-synaptic compartment and (2) carries a genomic “barcode,” a useful acronym is “SBARRO”: Synaptic barcode analysis with a retrograde rabies readout.

SBARRO-based inference of synaptic connectivity is scalable to millions of cells from individual experiments and can be adapted to different experimental systems (including both in vitro and in vivo systems) and sequencing platforms. In one embodiment, SBARRO may be paired with existing technologies for high-throughput single-cell RNA-seq, such as for example, 3′ end single-cell transcriptional profiling using Drop-Seq (Macosko, et al. Cell 161:1202-1214, 2015) or 10× (Zheng, et al. Nat. Commun. 8, 1-12, 2017), inferring networks and cell types simultaneously in vitro from cell culture or in vivo from adult mouse brains. A further embodiment uses SBARRO to infer synaptic connectivity within brains of any mammalian species. Another embodiment is directed to using SBARRO with high-throughput single-nucleus RNA profiling (Habib, et al. Nat. Methods 14(10):955-958, 2017), which supports analyses of both fresh-frozen tissue and large brains (where dissociating intact individual cells is typically challenging from large brains.) A further embodiment is directed to using SBARRO with the combination of in situ hybridization (Moffitt et al. PNAS 113(50):14456-14461, 2016) and in situ sequencing (Wang et al. Science 361(6400):eaat5691, 2018) of the RV barcodes to infer the neural networks in an intact brain (or in slices thereof), allowing for the retention of information about cellular anatomy and location. Another embodiment is directed to using SBARRO on other methods for “spatial transcriptomics” such as “Slide-seq” (Rodrigues and Stickels et al. Science 363(6434):1463-1467, 2019) or “High-Definition Spatial Transcriptomics” (Vickovic et al. Nat. Methods 16(10):987-990, 2019) that allow the capture of cellular and viral RNAs to be anchored to locations in space.

### Definitions

Unless defined otherwise, all technical and scientific terms used herein have the meaning commonly understood by a person skilled in the art to which this disclosure belongs. All terms used herein are intended to have their ordinary meaning in the art unless otherwise provided. The following references provide one of skill with a general definition of many of the terms used in this disclosure: Singleton et al., Dictionary of Microbiology and Molecular Biology (2nd ed. 1994); The Cambridge Dictionary of Science and Technology (Walker ed., 1988); The Glossary of Genetics, 5th Ed., R. Rieger et al. (eds.), Springer Verlag (1991); and Hale & Marham, The Harper Collins Dictionary of Biology (1991). As used herein, the following terms have the meanings ascribed to them below, unless specified otherwise.

All concentrations are in terms of percentage by weight of the specified component relative to the entire weight of the topical composition, unless otherwise defined.

As used herein, all ranges of numeric values include the endpoints and all possible values disclosed between the disclosed values. The exact values of all half integral numeric values are also contemplated as specifically disclosed and as limits for all subsets of the disclosed range. For example, a range of from 0.1% to 3% specifically discloses a percentage of 0.1%, 1%, 1.5%, 2.0%, 2.5%, and 3%, and all intervening percentages. Additionally, a range of 0.1 to 3% includes subsets of the original range including from 0.5% to 2.5%, from 1% to 3%, from 0.1% to 2.5%, etc. It will be understood that the sum of all weight % of individual components will not exceed 100%.

As used herein, “a” or “an” shall mean one or more. As used herein when used in conjunction with the word “comprising,” the words “a” or “an” mean one or more than one. As used herein “another” means at least a second or more.

The term “adaptor” refers a sequence that is added, for example by ligation, to a nucleic acid. The length of an adaptor may be from about 5 to about 100 bases, and may provide a sequencing primer binding site (e.g., an amplification primer binding site), and a molecular barcode such as a sample identifier sequence or molecule identifier sequence, preferably a unique identifier sequence. An adaptor may be added to 1) the 5′ end, 2) the 3′ end, or 3) both ends of a nucleic acid molecule. Double-stranded adaptors contain a double-stranded end ligated to a nucleic acid. An adaptor can have an overhang or may be blunt ended. As will be described in greater detail below, a double stranded adaptor can be added to a fragment by ligating only one strand of the adaptor to the fragment. The sequence of the non-ligated strand of the adaptor may be added to the fragment using a polymerase. Y-adaptors and loop adaptors are type of double-stranded adaptors.

By “alteration” is meant a change (increase or decrease) in the expression levels of a gene or polypeptide as detected by standard art known methods such as those described above. As used herein, an alteration includes a 10% change in expression levels, preferably a 25% change, more preferably a 40% change, and most preferably a 50% or greater change in expression levels.

By “amplicon” is meant a piece of a nucleic acid such as for example, DNA or RNA, that is the source and/or product of amplification or replication.

As used herein, the term “antisense strand” refers to a polynucleotide that is substantially or 100% complementary to a target nucleic acid of interest. For example, an antisense strand may be complementary, in whole or in part, to a molecule of mRNA (messenger RNA), an RNA sequence that is not mRNA (e.g., microRNA, piwiRNA, tRNA, rRNA and hnRNA) or a sequence of DNA that is either coding or non-coding. The terms “antisense strand” and “guide strand” are used interchangeably herein.

By “barcode” is meant a degenerate or semi-degenerate nucleic acid sequence that varies plasmid to plasmid or genome to genome. For example, any nucleic acid sequence that is highly variable across individual viral genomes or viral particles, such as but not limited to rabies virus or vesicular stomatitis virus (VSV), in a library. The barcode sequence may be a degenerate or a semi-degenerate sequence that is identifiable. For example, the barcodes may comprise identifiable degenerate sequences that have several possible bases in any of the positions of the nucleic acid sequence. A barcode may uniquely label or detect a single neuron. A barcode may also be used in sequencing to identify a genome. In an embodiment, a viral particle, comprising a genomic barcode refers to a “viral barcode,” such as a rabies virus particle (RVP).

A “cell culture” is a population of cells residing outside of an organism. These cells are optionally primary cells isolated from a cell bank, animal, or blood bank, or secondary cells that are derived from one of these sources and have been immortalized for long-lived in vitro cultures.

By “connectome” is meant the millions of points of contact between cells in the brain, including for example, neurons.

By “connectopathies” is meant disorders of neural or synaptic connectivity. For example, the total number of neurons and synapses may be normal, but may be connected in a less than ideal manner.

The phrase “in combination with” is intended to refer to all forms of administration that provide an inhibitory nucleic acid molecule together with a second agent, such as a second inhibitory nucleic acid molecule, where the two are administered concurrently or sequentially in any order.

In this disclosure, “comprises,” “comprising,” “containing” and “having” and the like can have the meaning ascribed to them in U.S. Patent law and can mean “includes,” “including,” and the like; “consisting essentially of” or “consists essentially of” likewise has the meaning ascribed in U.S. Patent law and the term is open-ended, allowing for the presence of more than that which is recited so long as basic or novel characteristics of that which is recited is not changed by the presence of more than that which is recited, but excludes prior art embodiments.

By “complementary” is meant capable of pairing to form a double-stranded nucleic acid molecule or portion thereof. In one embodiment, an antisense molecule is in large part complementary to a target sequence. The complementarity need not be perfect, but may include mismatches at 1, 2, 3, or more nucleotides.

By “corresponds” is meant comprising at least a fragment of a double-stranded gene, such that a strand of the double-stranded inhibitory nucleic acid molecule is capable of binding to a complementary strand of the gene.

By “decreases” is meant a reduction by at least about 5% relative to a reference level. A decrease may be by 5%, 10%, 15%, 20%, 25% or 50%, or even by as much as 75%, 85%, 95% or more and any intervening percentages.

By “exonuclease” is meant an enzyme that cleaves a polynucleotide chain from the end of the chain by removing the nucleotides one by one. In an embodiment of the disclosure, an exonuclease useful for selectively degrading linear DNA, as opposed to circular DNA, is RecBCD.

The term “expression” or “expressed” as used herein in reference to a gene means the transcriptional and/or translational product of that gene. The level of expression of a DNA molecule in a cell may be determined on the basis of either the amount of corresponding mRNA that is present within the cell or the amount of protein encoded by that DNA produced by the cell (Sambrook et al., 1989 Molecular Cloning: A Laboratory Manual, 18.1-18.88). Expression of a transfected gene can occur transiently or stably in a cell. During “transient expression” the transfected gene is not transferred to the daughter cell during cell division. Since its expression is restricted to the transfected cell, expression of the gene is lost over time. In contrast, stable expression of a transfected gene can occur when the gene is co-transfected with another gene that confers a selection advantage to the transfected cell. Such a selection advantage may be a resistance towards a certain toxin that is presented to the cell.

By “fragment” is meant a “portion” or part (e.g., at least 10, 20, 25, 50, 100, 125, 150, 200, 250, 300, 350, 400, or 500 amino acids or nucleic acids) of a protein or nucleic acid molecule that is substantially identical to a reference protein or nucleic acid and retains the biological activity of the reference.

The term “gene” means the segment of DNA involved in producing a protein; it includes regions preceding and following the coding region (leader and trailer) as well as intervening sequences (introns) between individual coding segments (exons). The leader, the trailer as well as the introns include regulatory elements that are utilized during the transcription and the translation of a gene. Further, a “protein gene product” is a protein expressed from a particular gene.

By “genomic library” is meant an entire genome of an organism, virus, bacteria, plant, or cell, or a collection of cloned DNA molecules consisting of at least one copy of every gene from a particular organism or cell.

By “high-throughput sequencing” is meant a sequencing technique that allows for large amounts of nucleic acids to be sequenced.

A “host cell” or “cell” is any prokaryotic or eukaryotic cell that contains either a cloning vector or an expression vector. This term also includes those prokaryotic or eukaryotic cells that have been genetically engineered to contain the cloned gene(s) in the chromosome or genome of the host cell.

By “hyper-diverse barcoded plasmid library” is meant a library of plasmids having unique, identifiable barcodes, where the diversity of barcodes may be in the hundreds of thousands to millions.

By “nucleic acid” is meant an oligomer or polymer of ribonucleic acid or deoxyribonucleic acid, or analog thereof. This term includes oligomers consisting of naturally occurring bases, sugars, and intersugar (backbone) linkages as well as oligomers having non-naturally occurring portions which function similarly. Such modified or substituted oligonucleotides are often preferred over native forms because of properties such as, for example, enhanced stability in the presence of nucleases.

By “operably linked” refers to a functional linkage between a regulatory sequence and a coding sequence, where a first polynucleotide is positioned adjacent to a second polynucleotide that directs transcription of the first polynucleotide when appropriate molecules (e.g., transcriptional activator proteins) are bound to the second polynucleotide. The described components are therefore in a relationship permitting them to function in their intended manner. For example, placing a coding sequence under regulatory control of a promoter means positioning the coding sequence such that the expression of the coding sequence is controlled by the promoter.

By “polyadenylation signal sequence” (poly(A) signal sequence) or “poly(A) tail” is meant a sequence of multiple adenosine monophosphates at the 3′-end of mRNA or cDNA. The poly(A) tail is particularly important for nuclear export, translation, and for stabilizing or protecting mRNA from nucleases.

By “portion” is meant a fragment of a polypeptide or nucleic acid molecule. This portion contains, preferably, at least 10%, 20%, 30%, 40%, 50%, 60%, 70%, 80%, or 90% of the entire length of the reference nucleic acid molecule or polypeptide. A fragment may contain 5, 6, 7, 8, 9, 10, 11, 12, 13, 14, 15, 16, 17, 18, 19, 20, or 21 nucleotides.

By “positioned for expression” is meant that the polynucleotide of the disclosure (e.g., a DNA molecule) is positioned adjacent to a DNA sequence that directs transcription and translation of the sequence (i.e., facilitates the production of, for example, a recombinant microRNA molecule described herein).

The term “promoter” as used herein refers to a sequence of DNA that directs the expression (transcription) of a gene. A promoter may direct the transcription of a prokaryotic or eukaryotic gene. A promoter may be “inducible”, initiating transcription in response to an inducing agent or, in contrast, a promoter may be “constitutive”, whereby an inducing agent does not regulate the rate of transcription. A promoter may be regulated in a tissue-specific or tissue-preferred manner, such that it is only active in transcribing the operable linked coding region in a specific tissue type or types.

The word “protein” denotes an amino acid polymer or a set of two or more interacting or bound amino acid polymers.

By “pseudotyped rabies virus” is meant a rabies virus (RV) in which its envelope gene has been replaced with an envelope gene from another species. For example, “EnvA-pseudotyped” where the RV envelope gene has been replaced with the envelope gene, EnvA of E. coli, which uses the TVA receptor for entry.

By “restriction enzyme” is meant an enzyme that recognizes particular DNA sequences, i.e., “restriction enzyme site” or “restriction site” and the restriction enzyme cleaves the DNA into fragments at or near the restriction enzyme site. Restriction enzymes allow a DNA molecule to be cut at a specific location.

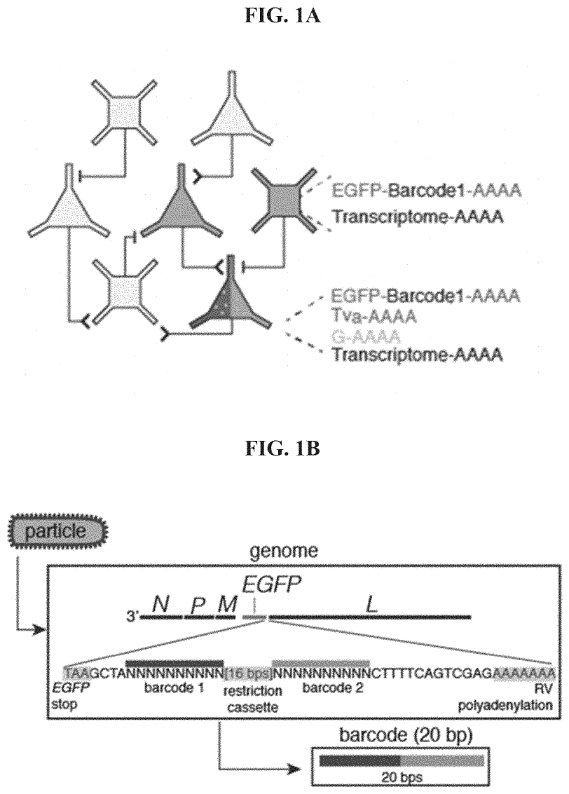

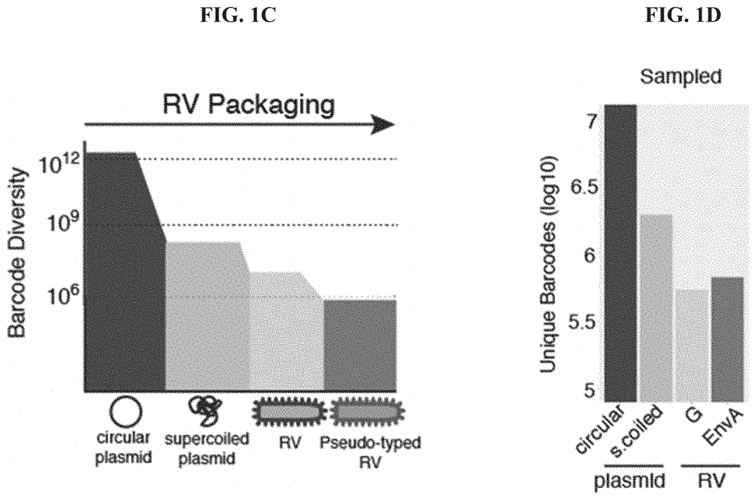

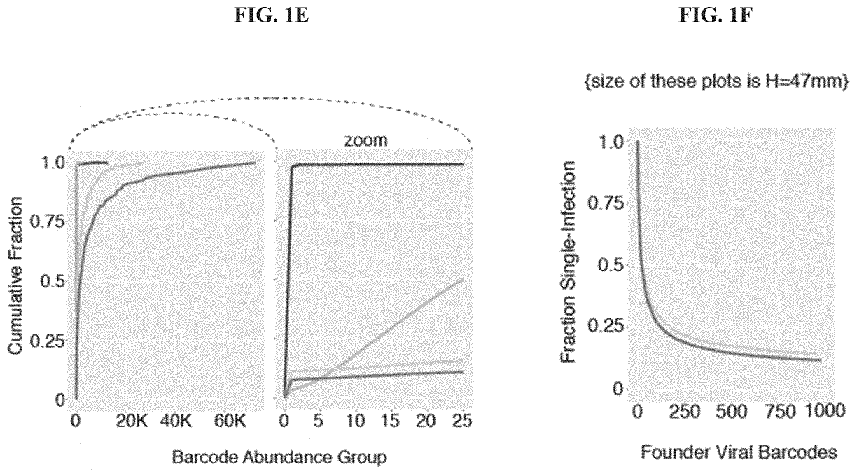

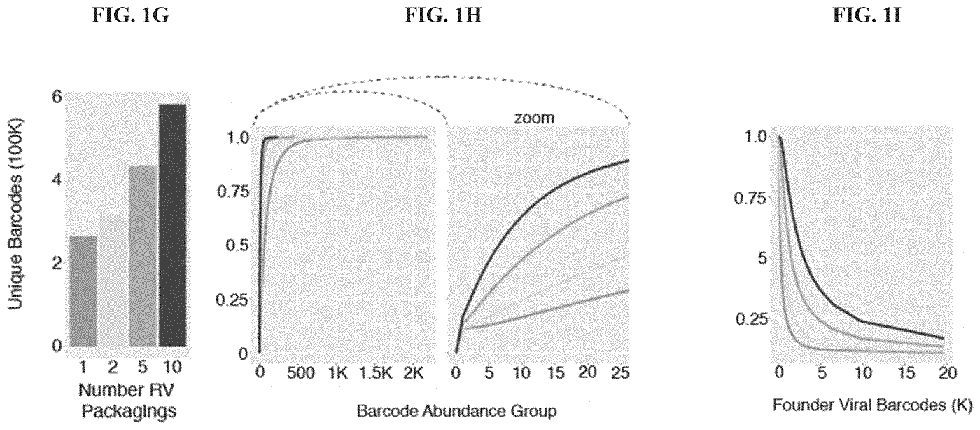

By “restriction enzyme cassette” or “restriction cassette” is meant a sequence containing a restriction enzyme site. The restriction cassette exemplified in FIG. 1B is 16 base pairs in length, but may be of any length that allows for a restriction enzyme to recognize and cut the DNA sequence at the appropriate restriction site, for example 6 base pairs to 20 base pairs in length or 6 base pairs to 12 base pairs in length.

By “RNA-seq” is meant RNA sequencing for detecting and quantifying messenger RNA molecules (mRNA) in a biological sample, which, for example, may be used to study cellular responses. A related term, “scRNA-seq” is single-cell RNA sequencing, which may be, for example, a droplet-based single-cell RNA-seq or “Drop-seq,” that is a sequencing technology for analyzing RNA expression in at least hundreds of thousands of individual cells in embodiments of the disclosure, but may alternatively use any other high-throughput sequencing platform.

By “RVdG” is meant glycoprotein (G)-deleted Rabies Virus (RV), where the gene encoding a glycoprotein of a “wild-type Rabies Virus (RV)” has been deleted. The RVdG prevents the spread of RV from presynaptic cells, thus restricting inferred networks to a single synaptic connection. For example, a “RVdG” may have replaced the gene encoding a RV glycoprotein with a gene encoding a “selectable moiety”, such as but not limited to a Green Fluorescent Protein (GFP) or an enhanced GFP (eGFP). Embodiments of the disclosure may use strains of RV, including but not limited to the B19, CVS, and N2C strains, that carry deletions of the glycoprotein “G” gene, i.e., “RVdG” in their genome that were engineered to transit only a single synapse. The deletion of the G gene enables not the spread of RV to be monosynaptically restricted, but also allows for pseudotyping and the selective primary infection of genetically defined neurons.

By “SBARRO” is meant Synaptic Barcode Analysis with a Retrograde Rabies Readout. “SBARRO” uses DNA sequencing to infer synaptic connectivity relationships among tens of thousands of transcriptionally-identified cell types. For example, SBARRO tracks Rabies Virus Particle (RVP) infectivity to infer synaptic connectivity.

A “selectable marker” that is suitable for use in the identification and selection of cells transformed or transfected with a cloning vector. Marker genes include genes that provide tetracycline resistance or ampicillin resistance, for example. Non-limiting examples of a selectable marker or detectable moiety include a fluorophore, an antibody resistance cassette, a capture molecule, a biotin molecule, streptavidin molecule, or an antigen.

By “selectable moiety gene” is meant a gene or nucleic acid sequence that is attached to a sequence of a gene of interest for identification and/or quantification. Non-limiting examples, of molecules encoded by a “selectable moiety gene” include a fluorophore, green fluorescent protein, enhanced green fluorescent protein, an antibody resistance cassette, an antigen, a capture molecule, a biotin molecule, a streptavidin molecule, or another selectable or identifiable molecule.

By “specifically binds” is meant a molecule (e.g., peptide, polynucleotide) that recognizes and binds a protein or nucleic acid molecule of the disclosure, but which does not substantially recognize and bind other molecules in a sample, for example, a biological sample, which naturally includes a protein of the disclosure.

“Starter cells” are the initial virus-infected cells or cells susceptible to infection. For example, hyper-diverse rabies virus barcode libraries infected “starter cells” or cells susceptible to infection, 1) to transduce the starter cell (e.g., the TVA receptor gene or the like) and 2) to spread (e.g., glycoprotein (“G”) or the like) from the starter cell into a larger number of cells, for example, “presynaptic” cells. “Starter cells” endow the cells with receptors that allow for infection by pseudotyped RV, where non-pseudotyped RV infect any cell.

The term “subject” is intended to include vertebrates, preferably a mammal. Mammals include, but are not limited to, humans and veterinary animals, which include but are not limited to dogs, cats, mice, horses, and the like.

By “synaptic connectivity” is meant the connection or transfer of signals between neurons or cells. A “retrograde synaptic connection” is the signaling from a postsynaptic target cell to the presynaptic neuron.

By “transcriptome” is meant all of the messenger RNA (mRNA) molecules expressed from the genes of an organism's RNA.

The term “transfection” or “transfecting” is defined as a process of introducing nucleic acid molecules into a cell. The introduction may be accomplished by non-viral or viral-based methods. The nucleic acid molecules may be gene sequences encoding complete proteins or functional portions thereof. Non-viral methods of transfection include any appropriate transfection method that does not use viral DNA or viral particles as a delivery system to introduce the nucleic acid molecule into the cell. Exemplary non-viral transfection methods include calcium phosphate transfection, liposomal transfection, nucleofection, sonoporation, transfection through heat shock, magnetifection and electroporation. In some embodiments, the nucleic acid molecules are introduced into a cell using electroporation following standard procedures well known in the art. For viral-based methods of transfection any useful viral vector may be used in the methods described herein. Examples for viral vectors include, but are not limited to retroviral, adenoviral, lentiviral and adeno-associated viral vectors. In some embodiments, the nucleic acid molecules are introduced into a cell using a retroviral vector following standard procedures well known in the art.

By “transformed cell” is meant a cell into which (or into an ancestor of which) has been introduced, by means of recombinant DNA techniques, a polynucleotide molecule encoding (as used herein) a protein of the disclosure.

By “unique molecular identifier” or “UMI” is meant short nucleic acid sequence that is identifiable in, for example, high-throughput sequencing techniques, such as but not limited to single-cell RNA-seq. The UMIs may be used to not only detect, but also to quantify. In embodiments of the disclosure, the UMIs are not viral barcodes.

By “vector” is meant a nucleic acid molecule, for example, a plasmid, cosmid, virus, or bacteriophage that is capable of replication in a host cell. In one embodiment, a vector is an expression vector that is a nucleic acid construct, generated recombinantly or synthetically, bearing a series of specified nucleic acid elements that enable transcription of a nucleic acid molecule in a host cell. Typically, expression is placed under the control of certain regulatory elements, including constitutive or inducible promoters, tissue-preferred regulatory elements, and enhancers.

By “viral genome” is meant the genomic information of a virus utilized to replicate themselves.

By “viral particle” or “virion” is meant an independent virus comprising genetic material, a protein coat or capsid, and with or without an envelope of lipids that surrounds the protein coat. A “rabies virus particle” is an enveloped independent virus containing the genetic material of the rabies virus.

By “viral vectors” or “viral-based vector” is meant a viral delivery means, for example, rabies virus. Additional viral vectors include, but are not limited to, adenovirus, adeno-associated virus (AAV), retroviral, lentiviral systems, hepatitis B virus, herpes simplex virus, and baculovirus.

A “virus” is an organism that cannot reproduce independently. Upon infection of an infection susceptible cell, a virus can direct the cellular machinery to replicate or produce more viruses. The genetic material of a virus may be either single-stranded or double-stranded RNA or DNA. The “rabies virus” (RV) is enveloped and has a single-stranded RNA genome with negative-sense. A wild type RV genome encodes five proteins: nucleoprotein (N), phosphoprotein (P), matrix protein (M), glycoprotein (G), and the viral RNA polymerase (L). An advantage of the rabies virus is that it spreads selectively between synaptically connected neurons and in the retrograde direction. Non-limiting examples of viruses used herein include a rabies virus and a vesicular stomatitis virus.

Molecular and synaptic network identities may be simultaneously inferred by high-throughput sequencing (i) a sample of the RNAs and (ii) the uniquely identified or barcoded RV transcripts from single cells. Cells that share RV barcodes participate in the same synaptic networks. Embodiments of the disclosure are directed to preparing libraries of RV carrying hyper-diverse collections of genomic barcodes, selectively infecting starter cells.

### Creating Libraries of RV Carrying Hyper-Diverse Collections of Genomic Barcodes

An embodiment of the disclosure is directed to a method of creating libraries of viruses (e.g., Rabies Virus (RV)) carrying hyper-diverse collections of genomic barcodes, in which distinct viral particles generally have distinct barcodes (FIG. 1). The diversity of the barcodes minimizes ambiguity about whether the sharing of viral barcodes (by distinct cells) represents an viral infection path (i.e., reveals that the cells are derived from the same synaptic network) rather than independent primary viral infections. Creating libraries with sufficient barcode diversity (and in uniform abundances) to statistically rule out cells in distinct networks sharing barcodes by chance is a major technical challenge. This difficulty is due to inefficiencies in how negative-stranded RNA viruses (such as RV) are created from DNA a process called “rescuing” or “packaging”. An embodiment of the disclosure overcomes this challenge by developing novel molecular methods for generating 1) barcoded DNA plasmids and 2) novel packaging conditions for creating and maintaining viral (e.g., RV) libraries with diverse barcodes. (See, e.g., FIG. 1)

One embodiment is directed to a method of constructing a hyper-diverse barcoded plasmid library by amplifying a reaction comprising: a forward primer, a reverse primer, and a template plasmid to produce an amplification product; purifying the amplification product; digesting the purified product with a restriction enzyme that recognizes the compatible restriction enzyme site to produce a digested product with overhangs or sticky ends; ligating the digested product to produce a circular barcoded plasmid; and selectively digesting linear DNA with an exonuclease. A further embodiment is directed to a forward primer and a reverse primer where both contain a compatible restriction enzyme site. Another embodiment is directed to a forward primer having a forward identifiable barcode sequence separated from the compatible restriction enzyme site by a forward linker sequence, and a forward region of protective bases or of sequence homology to the template plasmid, where the forward region is adjacent to the compatible restriction enzyme site. A further embodiment is directed to a reverse primer having a reverse identifiable barcode separated from the compatible restriction enzyme site by a reverse linker sequence, and a reverse region of sequence homology to the template plasmid, wherein the reverse region is adjacent to the compatible restriction enzyme site. In an embodiment, the identifiable barcode sequences (of the forward primer or reverse primer) may be separated from the compatible restriction enzyme site by 3 base pairs to 25 base pairs, 5 base pairs to 10 base pairs, or 5 base pairs. In another embodiment, the linker sequence (of the forward primer or reverse primer) may be 3 base pairs to 30 base pairs in length, 3 base pairs to 5 base pairs in length, or 5 base pairs in length. In a further embodiment, the region of sequence homology (of the forward primer or reverse primer) may be 18 base pairs to 200 base pairs in length. Yet another embodiment may be directed to the restriction enzyme site, where upon digestion with a compatible restriction enzyme that recognizes the restriction enzyme site, cleavage occurs such that overhangs or sticky ends are produced. The overhangs may be of any length that allows for circularization upon ligation. In an embodiment, the overhang is 4 base pairs. A further embodiment is directed to any restriction enzyme that cleaves in a manner that allows for overhangs or sticky ends, where PluTI or isoschizomers recognizing the same sequence. DpnI or other similar restriction enzyme may be used to digest any remaining template plasmid. Another embodiment may be directed to a T4 DNA ligase for ligating the overhangs or sticky ends. Ligation results in both intramolecular ligation which produces a circularized barcoded plasmid, or intermolecular ligation which produces a linear DNA. In yet a further embodiment, selectively digesting linear DNA without affecting the circularized barcoded plasmid may be accomplished using an exonuclease, where the exonuclease may be, for example, RecBCD.

In a further embodiment, a method of constructing a hyper-diverse barcoded plasmid library, comprises:
a) amplifying a template plasmid in an amplification reaction using a forward primer and a reverse primer, wherein the forward primer and the reverse primer each comprise from 5′ to 3′ a complementary region, a barcode, a linker, and a restriction enzyme site, where the restriction enzyme sites of the forward primer and the reverse primer generate 3′ compatible overhangs when cleaved;
b) generating double-stranded linear amplicons, each comprising the template plasmid sequence comprising barcodes at the termini of the amplicons;
c) digesting the amplicon with a restriction enzyme to produce a digested product comprising 5′ and 3′ overhangs;
d) ligating the digested product to produce a circular barcoded plasmid; and
e) selectively digesting linear DNA with an exonuclease.
   Another embodiment of the disclosure is directed to RecBCD as the exonuclease. A further embodiment may be directed to the barcode sequence (of the forward primer or the reverse primer) being separated from the compatible restriction enzyme site by 3 base pairs to 25 base pairs, or 3 base pairs to 5 base pairs, or 5 base pairs. Another embodiment may be directed to the linker sequence (of the forward primer or the reverse primer) of 3 base pairs to 30 base pairs in length, or 3 base pairs to 5 base pairs in length, or 5 base pairs in length. A further embodiment may be directed to the complementary region (of the forward primer or the reverse primer), where the complementary region is homologous to the template plasmid, being 3 base pairs to 20 base pairs in length, or 3 base pairs to 5 base pairs in length, or 5 base pairs in length. Another embodiment may be directed to the barcode sequence (of the forward primer or the reverse primer) of 3 base pairs to 30 base pairs in length, or 3 base pairs to 10 base pairs in length, or 10 base pairs in length.

A further embodiment may be directed to a library of hyper-diverse barcoded plasmids, where the hyper-diverse barcoded plasmid is a circular plasmid and comprises at least two identifiable barcode sequences separated from a restriction enzyme site by a linker sequence. The library comprises a multitude of the circularized barcoded plasmids, where the circularized barcoded plasmids comprise at least two identifiable barcode sequences separated from a restriction enzyme site by a linker sequence, at least three identifiable barcode sequences, at least four identifiable barcode sequences, and the like.

### Plasmid Barcoding

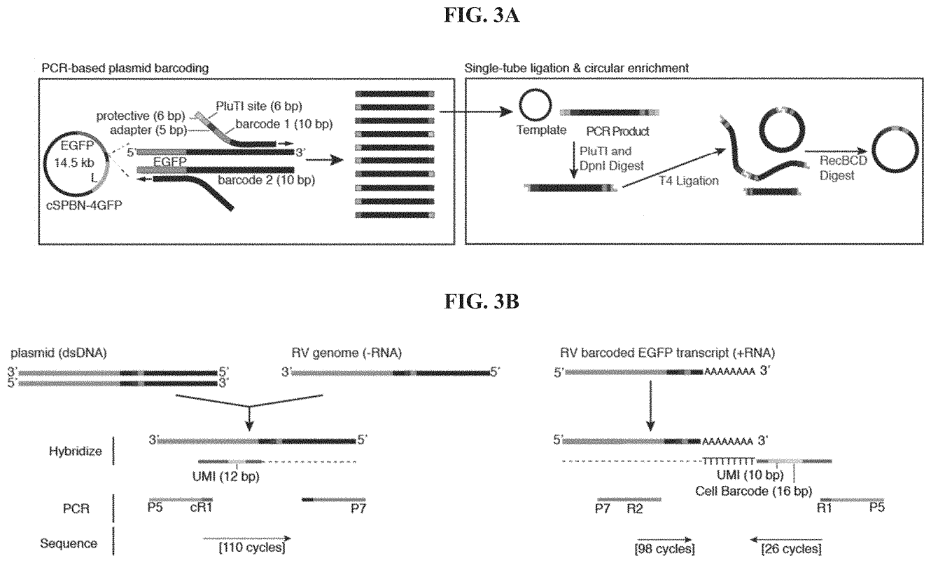

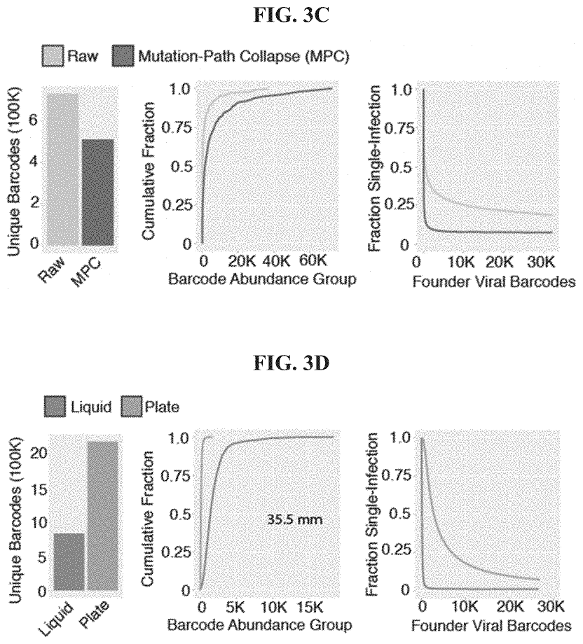

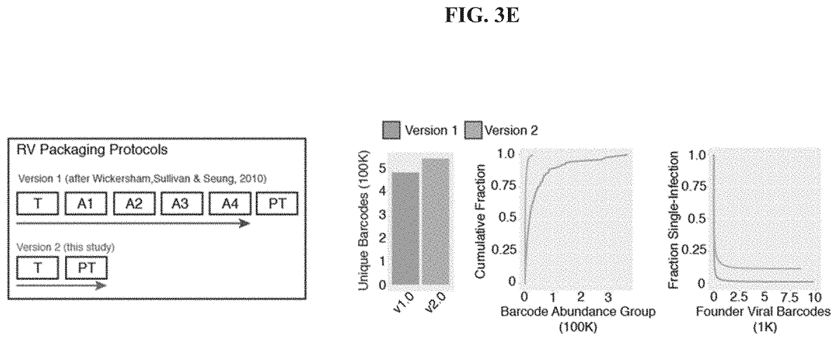

In one embodiment of the disclosure, a plasmid barcoding system was developed to generate microgram amounts of high-quality, circularized plasmid. This system, i.e., the “barcoding plasmid pipeline,” may introduce barcodes into any position of any plasmid of interest. An embodiment begins with a non-barcoded plasmid used as a template for PCR reactions in which random DNA sequences (barcodes) as well as shared restriction site cassettes are introduced through forward and reverse primers. (See, e.g., FIG. 3). Hundreds of micrograms of linear, double-stranded PCR amplicons encompassed the entire plasmid sequence with barcodes introduced on each terminal end of the amplified molecules. A further embodiment comprises circularizing the linear amplicons with a series of enzymes (such as in a single-tube), fusing the two terminal barcodes into a single barcode cassette, and eliminating any residual non-barcoded template plasmid.

In one embodiment, a template plasmid of recombinant rabies virus SPBN, generated from a SAD B19 cDNA clone (pSPBN) comprising a gene encoding an enhanced Green Fluorescent Protein (EGFP) transgene (RbV-GFP) and non-pseudotyped, glycoprotein-deleted rabies virus carrying tdTomato transgene (RbV-tdTom) were generated, with a retrograde tracer G-deleted non-pseudotyped rabies virus encoding tdTom CVS-N2c and CVS-B2c are highly pathogenic and less pathogenic subclones, respectively, of the mouse-adapted CVS-24 rabies virus.

### Rabies Virus Rescuing or Packaging

Another embodiment is directed to a shorter RV rescue method of about 9 days compared to the typical RV rescue protocols which are more than 1 month and involve a series of “amplification” steps that are detrimental to maintaining diverse genomic barcodes. The shorter RV rescue method involves no amplification steps or a minimal number of amplification steps which preserves barcode diversity and leads to a more uniform barcode distribution. One of skill in the art appreciates that the following methods are generally applicable to a variety of viruses, and that RV is used as one exemplary virus.

In an embodiment of the disclosure, a rabies virus particle genome may comprise of a 3′ to 5′ linear, nucleic acid sequence having a RV nucleoprotein gene, a RV phosphoprotein gene, a RV matrix protein gene, a selectable moiety gene, a viral barcode gene, and a RV polymerase gene, wherein the viral barcode gene is positioned within a transcribed sequence comprising at least two identifiable barcode sequences separated by a restriction enzyme cassette and a polyadenylation signal sequence. Any of the genes may be an endogenous or transgene. A selectable moiety gene may encode a fluorophore, an antibody resistance cassette, a capture molecule, a biotin molecule, streptavidin molecule, or an antigen, or combinations thereof. In a further embodiment, the selectable moiety gene may encode a fluorophore, such as but not limited to, an enhanced green fluorescent protein.

Yet another embodiment may be directed to a rabies virus genome, comprising: a 3′ to 5′ linear, nucleic acid sequence encoding a rabies virus (RV) nucleoprotein, a RV phosphoprotein, a RV matrix protein, a barcode, and a RV polymerase, where the barcode comprises a restriction enzyme cassette. Any of the one or more genes may be an endogenous gene or transgene. The rabies virus genome may further comprise a selectable marker or detectable moiety, where the detectable moiety is a fluorophore, and the fluorophore may be, for example, a fluorescent protein or the like, such as but not limited to a green, red, or yellow fluorescent protein. Another embodiment may be directed to a rabies viral genome as described here, where the restriction enzyme cassette divides the barcode into two halves. In one embodiment, a viral particle may comprise the viral genome described herein, including but not limited to the rabies virus genome.

Yet a further embodiment may be directed to a method of inferring synaptic connectivity, comprising:
a) contacting one or more starter cells susceptible to infection with a library of viral particles each comprising a rabies genome of Claim 1;
b) replicating the virus particle within the one or more infected starter cells;
c) allowing the virus to infect one or more additional cells, each synaptically connected to the starter cell;
d) optionally sorting the cells to select the rabies virus infected cells;
e) creating single-cell RNA-seq libraries from the infected cells;
f) high-throughput sequencing of the single-cell RNA-seq libraries to identify RNA sequences and viral barcodes present within each cell; and
g) inferring synaptic connectivity of cells based on the sharing of at least one identifiable barcode, wherein cells that share an identifiable barcode are inferred to be in the same synaptic network.

In another embodiment, the method of inferring synaptic connectivity may further comprise identifying cell types or cell type information from the identified RNA sequences. Another embodiment may be directed to a method of simultaneously or sequentially, in any order, inferring synaptic connectivity and identifying cell types or cell type information. In yet a further embodiment, the identifiable barcode comprises a selectable marker or detectable moiety, which includes but is not limited to, a fluorophore, an antibody resistance cassette, a capture molecule, a biotin molecule, streptavidin molecule, or an antigen. In another embodiment, the selectable marker or detectable moiety of the rabies virus genome may include, and is not limited to, a fluorophore, an antibody resistance cassette, a capture molecule, a biotin molecule, streptavidin molecule, or an antigen.

Another embodiment may be directed to a barcode gene that has a 3′ to 5′ linear, nucleic acid sequence comprising: at least two identifiable barcode genes and a selectable moiety gene, wherein the at least two identifiable barcode genes comprises a barcode gene positioned within a transcribed sequence comprising at least two identifiable barcode sequences separated by a restriction enzyme cassette.

A further embodiment may be directed to polynucleotide encoding a barcode, comprising a restriction enzyme cassette, wherein the restriction enzyme cassette separates the barcode into two equal or unequal halves. Another embodiment of the polynucleotide encoding a barcode may further comprise a selectable marker or detectable moiety, where the detectable moiety is a fluorophore, and the fluorophore is a fluorescent protein.

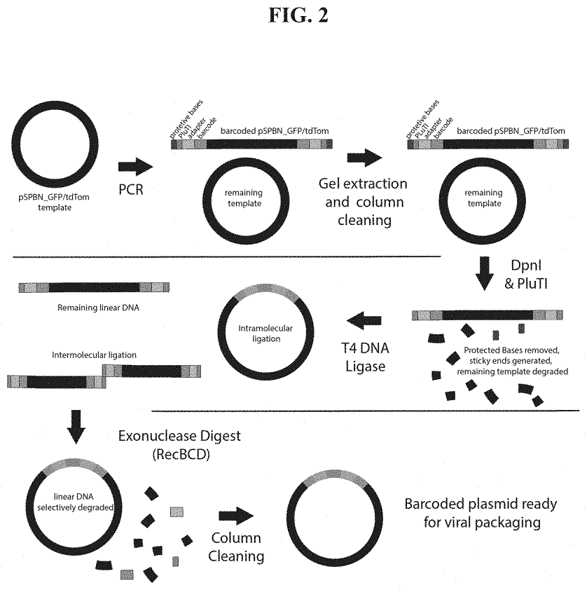

In one embodiment, Rabies Virus cDNA may be barcoded with two 10 bp barcodes. (See, e.g., FIG. 2). For example, a pSPBN_GFP/tdTom template may undergo PCR to form a barcoded pSPBN_GFP/tdTom comprising protective bases, a restriction enzyme site, e.g., PluTI, an adapter, and barcode at both ends of the plasmid fragment where the protective bases are positioned at the outermost ends in a linear order such that the barcodes are positioned at the innermost ends, maintaining the remaining template. Gel extraction and column cleaning may be performed. Afterwards, digest with restriction enzymes that can degrade the remaining template and remove the protected bases on each end as well as generate sticky ends. For example, the restriction enzymes DpnI and PluTI may be used, or any isoschizomers, i.e., restriction endonucleases that recognize the same sequence, that result in sufficient overhang to produce the restriction enzyme sticky ends including but not limited to, KasI, DinIEgeI, EheI, KasI, Mly113I, NarI, sfol, sspDI, NarI, and the like. In one embodiment, a restriction enzyme for generating sticky ends utilizes two copies of its recognition sequence for cleavage to occur, i.e., utilizes two or more sites for cleavage. The plasmid fragment comprising two barcodes at the outermost positions with the adapters adjacent to each barcode may be ligated (e.g., using T4 DNA ligase) such that the restriction enzyme sticky ends perform intramolecular ligation. Alternatively, intermolecular ligation between different linear fragments may occur at the restriction enzyme sticky ends, then linear DNA may be selectively degraded by exonuclease digest with, for example, RecBCD, where after column cleaning a barcoded plasmid is prepared for viral packaging. In one embodiment the restriction digest, ligation, and exonuclease digest may be performed stepwise in a single tube. Another embodiment may be directed to multiple reactions each in its own contained area, including but not limited to, wells of a multiarray plate.

Another embodiment may be directed to barcoding primers for a pSPBN plasmid (SAD-19 strain), where the primers may include the following, where ‘N’ represents the barcode position.

TABLE 1

| Primer | Sequence |
|---|---|
| MW_tdTom_v9_PluTI_ | 5′-ACCTGTGGCGCCACTGCNNNNNNNN |
| 5adpt_Forward | NNCTTTTCAGTCGAGAAAAAAACATTAG |
| | ATCAGAAGAACAAC-3′ SEQ ID |
| | NO: 3 |
| MW_tdTom_v9_PluTI_ | 5′-ACCTGTGGCGCCACCGGNNNNNNNN |
| 5adpt_Reverse | NNTAGCCTACTTGTACAGCTCGTCCATG |
| | CCGTACAGGAAC-3′ SEQ ID NO: 4 |
| MW_GFP_v9_PluTI_ | 5′-ACCTGTGGCGCCACCGGNNNNNNNN |
| 5adpt_Reverse | NNTAGCTTACTTGTACAGCTCGTCCATG |
| | CCGAGAGTGA-3′TC SEQ ID NO: 5 |

### Packaging Barcoded Rabies Virions

In one embodiment, barcoded rabies virus genomes of the CVS-N2cΔG strain may be packaged into EnvA-pseudotyped virions. In a further embodiment, glycoprotein-deleted rabies virions may be generated from cDNA to produce high diversity barcoded rabies virus libraries. Packaging barcoded rabies virions comprises recovering barcoded virions from cDNA, pseudotyping with EnvA glycoprotein coat, and collecting the pseudotyped virions.

Synaptic Barcode Analysis with a Retrograde Rabies Readout (SBARRO) System

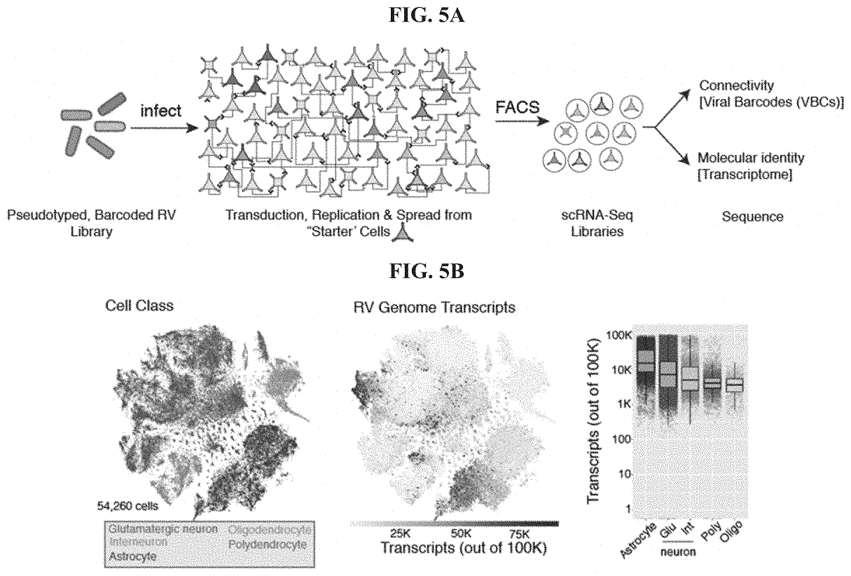

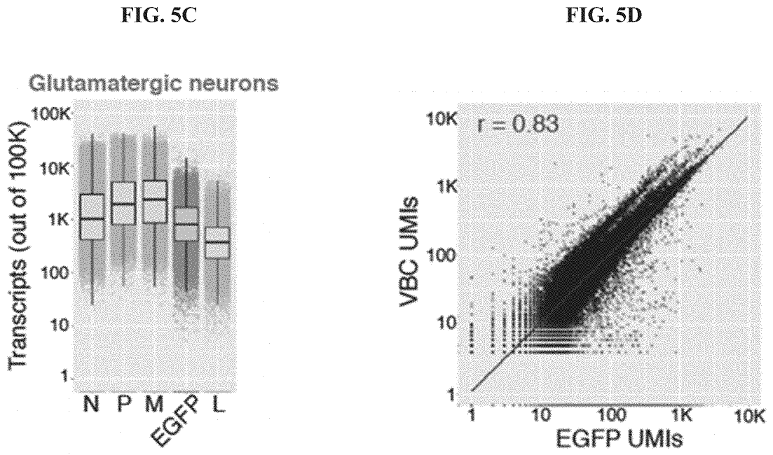

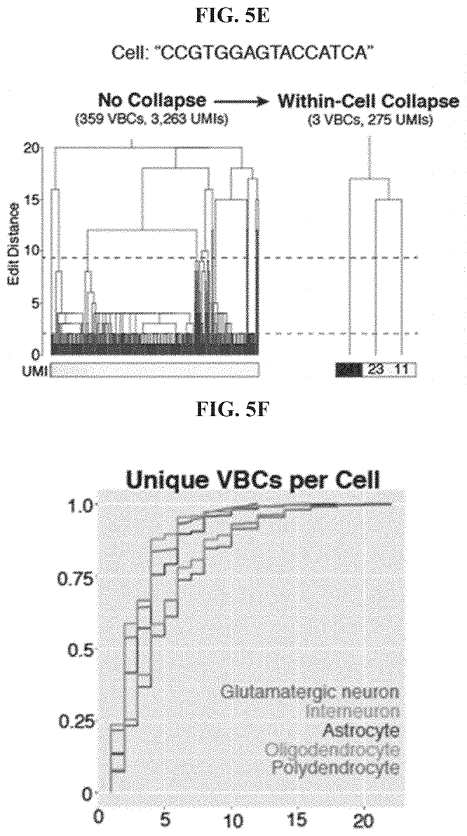

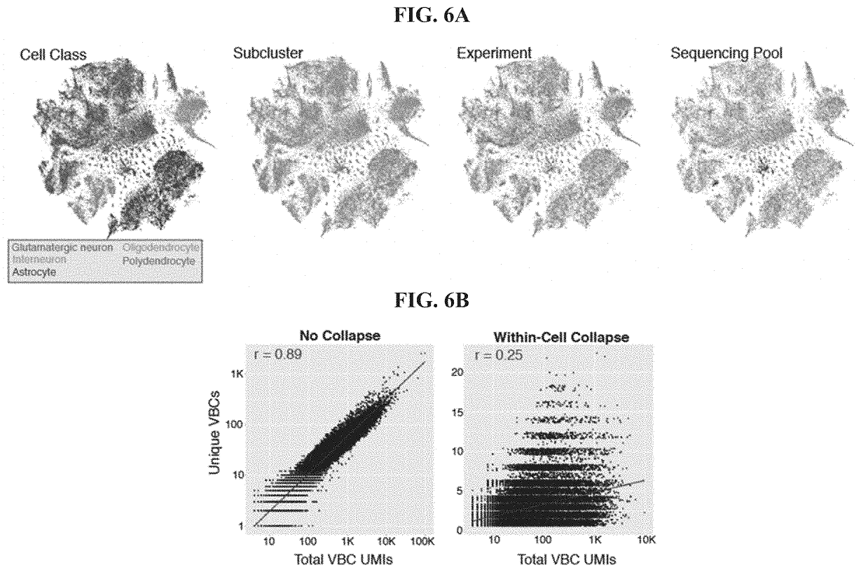

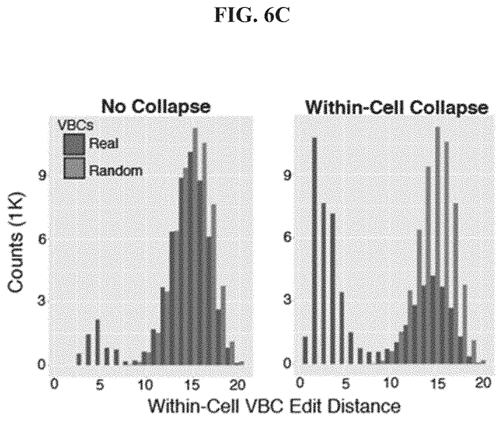

SBARRO is based on novel molecular and virology techniques for engineering Rabies Virus (RV) libraries carrying hyper-diverse genomic barcodes (see, e.g., FIG. 5). Molecular and synaptic network identities may be simultaneously inferred by sequencing the nuclear transcriptome and barcoded RV transcripts from single or individual cells. Cells that share RV barcodes participate in the same synaptic networks. In one embodiment, the SBARRO system comprises: selectively infecting “starter” cells with a barcoded RV library. The number and molecular identity of starter cells may be controlled by the experimenter. The infected presynaptic cells are a subset of the total ensemble of cells that make synaptic inputs onto the “starter” cell.

In an embodiment of the disclosure, SBARRO-infected networks may be dissociated into single or individual cells. The RV-infected population may be enriched from the uninfected (and uninformative) cell population using fluorescent activated cell sorting (FACS). Single-cell transcriptome libraries may be created from the RV infected cells. RV barcodes and genome-wide transcriptomes may be independently amplified and sequenced from the resulting cDNA. RV barcodes may be computationally extracted from sequencing data.

A further embodiment is directed to using SBARRO (combined with 10× single-cell transcriptomics) to sequence synaptic networks from about 60,000 cells derived from embryonic mouse cortex (about 60,000) cells) and from adult mice in vivo (about 45,000 cells). Barcoding alternative strains that exhibit enhanced synapse-transmitting in vivo (e.g., N2C or CVS) and creating 2nd generation “helper” viruses (like the adeno-associated viruses (AAVs) that deliver the avian retroviral receptor, TVA, and glycoprotein G genes to define starter cells) produce expression patterns that may be more easily measured through single-cell transcriptomics.

Another embodiment is directed to computational methods used to assign cells sharing the RV barcodes to networks. The molecular identities of each individual cell (including starter/presynaptic status) may be distinguished by their RNA expression patterns.

### Applications

Another embodiment of the disclosure is directed to using the SBARRO system in research to ascertain synaptic connectivity relationships, capturing such relationships as a high-dimensional, quantitative phenotype that is amenable to statistical comparison for research. In experimental animals, SBARRO-based comparisons may be used to infer similarities and differences in synaptic networks, including, but not limited to, the following example comparisons: 1) inter-species: 2) intra-species genotypes (including disease models) and 3) developmental state or 4) the effect of therapeutics in animal models.

In an embodiment, the SBARRO system may also be used to infer how variation in the human genome affects synaptic connectivity relationships between human brain cell types. Cultures of human brain cell types from one or more individuals could be differentiated from induced pluripotent stem cells (iPSCs) and grown in vitro (including brain “organoids”). The resulting synaptic networks (and molecular identity of individual cells, including genotype) could be inferred with SBARRO.

A further embodiment may be directed to clinical applications, where many potential medicines have unknown effects on synaptic connectivity, and such effects could be pathological or part of their therapeutic action. SBARRO-based assays of synaptic connectivity may be used in animal models or in vitro neuronal/organoid cultures to evaluate the effects of candidate therapeutics on a wide variety of synaptic connections.

In another embodiment, the methods may correlate mutations and illnesses with their effects on synaptic connectivity. Neuropsychiatric conditions are proposed to involve disturbances in synaptic connectivity (called “connectopathies”). The inventive SBARRO-based assays of synaptic connectivity using induced pluripotent stem cell (iPSC)-derived cultures of brain cells, or animals with mutations analogous to those in human patients, may be helpful in identifying specific connectivity deficits, generating therapeutic hypotheses that may in principle be addressed by such approaches as (i) targeted deep brain stimulation of the relevant cell populations or circuits: (ii) medicines that affect the physiological properties of specific cell populations.

A further embodiment is directed to the application of the SBARRO system for (1) assessing the effects of therapeutics and/or mutations on synaptic connectivity, and/or (2) ascertaining the specific connectivity defects that are present in a specific disorder, or in patients with a specific mutation, in order to develop effective therapeutic hypotheses. An embodiment of the disclosure may be directed to the molecular and synaptic network status of individual cells using single-cell transcriptomic methods like Drop-seq and 10× for analysis or obtaining a “read out.” Platforms for reading out SBARRO are flexible, however, and could also include in other embodiments, in situ sequencing and in situ hybridization methods which allow synaptic networks and cell types to be identified within intact brain tissue. Further embodiments provide for methods using SBARRO on “spatial transcriptomics” allowing for the determination of the locations of genes, the entire transcriptome, in a cell or tissue to be inferred by sequencing. For example, the “spatial transcriptomics” techniques include, but are not limited to, “Slide-seq” or “High-Definition Spatial Transcriptomics” (H DST) which enable both gene expression and spatial or positional information in tissues. Briefly, Slide-seq uses a single layer of DNA-barcoded beads on a glass slide to capture mRNAs released from a tissue section physically placed on top of the glass slide; whereas, HDST uses microbeads in a microwell array. The combination of SBARRO with these techniques allow the capture of cellular and viral RNAs and their anchoring to spatial positions.

Another embodiment may be directed to a CRISPR screening method, where the knockout or over-expression of a gene is in culture or in vivo and the cells that fail to survive a selection process screening may be used to identify genes that are essential for growth or survival under certain conditions when compared to a non-selected control. Yet a further embodiment may utilize the library of hyper-diverse barcode plasmids described here for screening purposes related to the effect of the manipulated genes on the size or cellular makeup of synaptic networks. The CRISPR state of the manipulated cell can be read out simultaneous to 1) molecular identity and 2) synaptic network composition.

### EXAMPLES

The following examples illustrate specific aspects of the instant description. The examples should not be construed as limiting, as the example merely provides specific understanding and practice of the embodiments and its various aspects.

### Example 1: Plasmid Barcoding

Rabies virus cDNA was prepared with two 10 base pair barcodes. (See, FIG. 2.) The reagents for the PCR reactions include: rabies virus genomic cDNA template freshly diluted to 0.4 ng/μl, barcoding primers at a working concentration of 10 μM, Q5® High-Fidelity 2× Master Mix DNA polymerase (New England Biolabs: M0492L), and ultrapure “Type 1” water (e.g., ISO 3696) (Millipore Sigma; Burlington. Mass. USA) in 96 well plates.

The barcoding primers for the pSPBN plasmid (SAD-19 strain), where ‘N’ represents a barcode position, are provided in TABLE 1.

The optimized PCR reaction was performed as follows:

TABLE 2

| | Per 25 μl | | Per PCR plate |
|---|---|---|---|
| Reagent | reaction | | (100 25 μl reactions) |
| Q5 ® Master Mix | 12.5 | μl | 1250 μl |
| | | | (1 full tube of Q5) |
| Forward Primer (10 μM) | 1.25 | μl | 125 μl |
| Reverse Primer (10 μM) | 1.25 | μl | 125 μl |
| Template Plasmid | 5 | μl | 500 μl |
| (pSPBN_GFP/tdTom), 2 | | | |
| ng per rxn at 0.4 ng/μl | | | |
| Water | 5 | μl | 500 μl |

Reactions were carried out at a volume of 25 μl to 30 μl. Larger volumes would result in the PCR to fail. The template aliquots were stored at −20° C. and at a higher concentration than the working stock of 4 ng/μl in order to avoid any rapid degradation or negatively affected yields at the lower storage concentration. A storage of 10 ng/μl and higher worked well. Template degradation was found to be the largest impediment to effective PCR amplification. If yields dropped, the template was re-diluted. Excessive freeze/thaw of the stock was avoided and the plasmid quality was checked periodically via agarose gel.

The thermocycler protocol that was run as follows.

TABLE 3

| Cycle Step | Temperature | Time | Cycles |
|---|---|---|---|
| 1-Initial Denaturation | 98° C. | 30 sec | 1 cycle |
| 2-Denaturation | 98° C. | 10 sec | |
| 3-Annealing | 72° C. | 30 sec | |
| 4-Extension | 72° C. | 450 sec (7.5 min) | |
| 5-Repeat Cycle Steps | Go to Steps 2-4 | | 35 cycles |
| 6-Final Extension | 72° C. | 2 mins | 1 cycle |
| 7-Hold | 4° C. | Indefinite | |

After the PCR has run to completion, all of the reactions were combined into a 5 ml PCR clean, LoBind Eppendorf® tube and stored at 4° C. up to 3 days. If there was a white precipitate at the bottom of the tube, the volume was resuspended before proceeding with the gel separation and purification.

The reagents and equipment used for gel separation and purification included: TAE buffer (Tris-acetate-EDTA; Concentration 1×, pH8.0; Sigma Aldrich); low gelling temperature agarose (A9414; Sigma-Aldrich); blue light transilluminator (UltraSlim Blue Light Transilluminator, Transilluminators.com): 500 mL Erlenmeyer flasks; microwave; Gel Loading Dye, Purple (6×), no SDS (B7025S; New England BioLabs): 1 kb Plus DNA Ladder (Invitrogen™); Scapel/razor blade: 50 mL Falcon Tubes, Large gel rig: Lab tape, Invitrogen™ SYBR™ Safe DNA Gel stain (S33102: ThermoFisher Scientific); Zymoclean™ Gel DNA Recovery Kit (Zymo Research); DNA Clean & Concentrator™-25 (Zymo Research); and optionally, 1.5 ml PCR clean, LoBind Eppendorf® tubes.

In order to separate the desired band, the PCR product was run on a 0.7% TAE gel with low gelling temperature agarose. Lab tape was used on gel combs to create large mega wells, leaving one well on the comb for the 1 kb Plus DNA ladder. For smaller purifications (i e, up to 48×25 μl reactions), the smaller gel casting rig was used (Owl™ Easy Cast™ B2 Mini Gel Electrophoresis System; Thermo Scientific™). For larger purifications, multiple gels were run on the smaller system using the Bio-Rad® Sub-Cell® GT Cell apparatus (1 plate PCR reactions) or run 3 plates worth of PCR reactions at a time with the Owl™ A3-1 Large-Gel Electrophoresis System (Thermo Scientific™).

The TAE gel was poured into the small and large gel electrophoresis rigs by adding 150 ml (small gel rig) or 300 ml (large gel rig) TAE to a 500 ml Erlenmeyer flask. To account for evaporation when heating, 154 ml or 310 ml of TAE was measured for the small or large rig, respectively. The boron in TBE inhibits downstream ligation. Therefore, if there was any glassware that may have been used with TBE, the glassware, such as for example, a graduated cylinder, was rinsed thoroughly with deionized water before use. For extra-large rigs, two 500 mL Erlenmeyer flasks were filled with 350 mL TAE. While vigorously stirring the TAE with a stir bar, low gelling agarose was added slowly in small amounts to avoid the formation of clumps. Low gelling agarose was added as follows: 1.05 g for a small gel; 2.10 g for a large gel; and 2.45 g×2 for an extra-large gel. All large clumps were broken up before more of the agarose was added. Before the TAE/agarose was microwaved on high for 2 minutes for the small and large gels, and 2.5 minutes for the extra-large gel, the stir bar was removed from the flask. The flask was removed and the contents in the flask were swirled around before microwaving for another 30 seconds to 1 minute for the small and large gels, and an additional 1.25 minutes. After the second round of microwaving, the flask was examined for foam on the surface of the agarose or floating agarose “seeds.” If present, another round of microwaving was needed. For the extra-large gel, the first 350 mL of TAE/agarose was poured into a 1 L flask and slowly stirred. The microwaving process was repeated with the second flask of 350 mL TAE/agarose. Both were combined and allowed to cool for 5-10 min at room temperature SYBR™ Safe DNA Gel stain at a concentration of 10,000× was added to a small gel (15 μl), a large gel (30 μl), and an extra-large gel (70 μl). After the TAE/agarose with gel stain cooled to a temperature cool enough to handle and pour but not so cool as to result in “seeds” of agarose reforming when poured. The gel was poured into the appropriately sized casting rig such that the rig was as level as possible. Any large bubbles in the gel were popped. The gel was cooled at 4° C. for 1 hour. If the formed gel was not used immediately. TAE buffer was covered the gel to prevent the gel from drying out. To avoid tearing any wells in the gels, particularly for the agarose gels in the large and extra-large casting rigs which may adhere to the panels on the short ends of the gel, the seal was released and the end of the rig was not pulled off. For both the large and extra-large gels, a metal spatula was used to separate the gel from the rig along the rubber gel interface before freeing the gel from the casting rig.

The gel tray was placed in the gel box, and the gel box was filled with TAE buffer. The small tray used about 800 mL of TAE buffer; the large tray used about 1200 mL to 1500 mL; and the extra-large tray used about 3.5 L to 4 L of TAE buffer. Prior to loading the gel, the combs were removed. Any excess agarose blocking the well was removed. Loading dye was added to each reaction such that final concentration was 1 in 6 (Gel Loading Dye. Purple (6×), no SDS) was added to the previously combined PCR reactions and mixed thoroughly (i.e., pipetted up and down about 10 times) to ensure there was no precipitate that had settled immediately before loading the gel. The sample of combined PCR reactions with loading dye was pipetted very slowly into the mega wells without overloading. In a separate well, the 1 kb Plus DNA Ladder or lambda HinDIII digest was added as a reference for estimating the band size. The gels were run for about 1.5 hours at 70V (small), 100V (large), and 120V (extra-large) until the blue shadow of the loading dye was about an inch or more away from the wells.

In order to cut the band and purify the DNA, a blue light transilluminator was used for gel cutting. Prior to cutting, a 1×50 mL Falcon® tube was rinsed, weighed per PCR plate's worth of reactions to the nearest milligram, and weight recorded. The brightest agarose band visible was cut out avoiding any excess agarose and placed in the Falcon® tube. The weight of the gel was calculated. The Zymoclean™ Gel DNA Recovery Kit was used with the following modifications. A modified cleaning protocol was used. An appropriate amount of agarose dissolving buffer (3× mass of the gel) was added to the gel to dissolve the agarose gel at 37° C. for about 30 minutes and periodically mixed by inversion. The “gel only” mass of water was added to the tube and mixed by inversion. This mixture was added to each column (780 μl) and spun at 15,000 g for 30 seconds. The flow was discarded. The reactions were loaded and spun through the columns as necessary. Once all of the reactions with the DNA binding buffer were spun through, new collection tubes were used. Wash buffer (215 μl) was added to each column and spun at 15,000 g for 30 seconds. The flow was discarded and another 215 μl of wash buffer was loaded on each column and spun at 15,000 g for 1 minute. The columns were then placed in 1.5 Eppendorf® tubes. The desired total elution volume was divided in two. Water was added and allowed to sit on the column for 5-10 minutes before spinning the sample at 15,000 g for 30 seconds. Usually 2×15 μl of water was used for elution. The second volume of water (e.g., 15 μl) was added and allowed to sit on the column for 5-10 minutes before spinning the sample at 15,000 g for 30 seconds. All of the reactions that corresponded to an initial PCR plate were pooled.

A second clean was performed on the DNA with the DNA Clean & Concentrator™-25 columns. All spins were performed at 15,000 g. Two volumes of DNA binding buffer were added to each DNA pool and pipetted up and down to mix. Up to 4 reactions per column per spin (about 770 μl) was added carefully to avoid spilling the reactions. The columns were spun at 15,000 g for 30 seconds. The flow through was discarded, another 215 μl of wash buffer was added, and spun at 15,000 g for 30 seconds. The collection tubes were changed and spun for 2 minutes. The columns were placed into 1.5 mL Eppendorf® tubes. The desired total elution volume was divided by two (about 60 μl) and eluted with water by allowing the first volume to sit on the column for 5-10 minutes before spinning. The same elution steps were repeated for the second volume of water. The elution from the columns containing DNA from the same PCR plate were pooled. The DNA concentration was evaluated by Nanodrop Nucleic Acid Quantification. The average PCR plate yielded about 25 μg of DNA (greater than 200 ng/μl if each PCR plate was eluted in 120 μl water).

### Example 2: Single Tube Processing

The reagents used for single tube processing of the restriction digest, ligation, and selective exonuclease digest included: PCR double cleaned DNA; PlutI-HF (NEB); DpnI (NEB); CutSmart® Buffer 10×; T4 Ligase (NEB, 2,000,000 U/mL); ATP; NEB Buffer 4; RecBCD/exoV (NEB): DNA Clean & Concentrator™-5 (Zymo Research); Molecular grade water; and 96 well plates which hold at least 200 μl per well. The quality of the DNA was paramount for this reaction. PCR DNA was of a high quality with few contaminants and the freeze/thaw cycles were minimized. Only fresh, non-degraded ATP was used for the ligation. As ATP degrades to AMP, it can catalyze cutting activity by T4 ligase. ATP was stored at −80° C.

### PluTI Restriction Enzyme Digest

In order to create the sticky ends for ligation. PluTI was used. Since the template was isolated from bacteria, the was methylated and DpnI selectively digested it. However, other restriction enzymes which are functionally equivalent to PluTI and DpnI, such as for example, isoschizomers which recognize the same sequence, may be used. The barcoded DNA amount was as indicated in the below reaction mixture and not in excess. The recommended amount of barcoded DNA was particularly important to adhere to in order to avoid the formation of concatemers over intra-molecular ligation which would occur at higher DNA concentrations. The restriction enzyme reaction was incubated at 37° C. for 1 hour and heat inactivated at 80° C. for 20 minutes. The restriction enzyme digest reaction was as follows:

TABLE 4

| Reagent | Volume for 50 μl reaction |
|---|---|
| PluTI | 0.5 μl |
| DpnI | 0.25 μl |
| CutSmart ® | 5 μl |
| Buffer (10×) | |
| Barcoded DNA | 500 ng |
| Water | To 50 μl |

The Master Mix reaction for 98 reactions was as follows:

TABLE 5

| Reagent | Volume for 50 μl reaction |
|---|---|
| PluTI | 49 μl |
| DpnI | 24.5 μl |
| CutSmart ® | 490 μl |
| Buffer (10×) | |
| DNA | 218.54 μl |
| Water | 4,117.96 μl |

### Ligation Reactions

The goal was to re-circularize as much DNA as possible. Ligase and ATP were added directly to the completed PluTI digest reactions. The ligation reaction was incubated at 4° C. for 2 hours followed by heat inactivation at 65° C. for 20 minutes. If a ligation spike was performed on an entire plate, 3 additional reactions of master mix were made and included. If ligation rates were low, the DNA was heated for 5 minutes at 65° C. prior to ligation. If this was done, ligation rates were improved following this method up to 5%. The ligation reaction was as follows:

TABLE 6

| Reagent | Volume for 50 μl Reaction |
|---|---|
| T4 ligase | 0.1 μl |
| (200U) | |
| ATP | 5 μl |

The master mix ligation reaction was as follows:

TABLE 7

| Reagent | Volume for 50 μl Reaction |
|---|---|
| T4 ligase | 10 μl |
| (200U) | |
| ATP | 500 μl |

Selective Exonuclease Digestion with RecBCD exoV

The purpose of the RecBCD digest was to enrich for circular species. The exonuclease digestion was as follows:

TABLE 8

| Reagent | Volume for 50 μl Reaction |
|---|---|
| Original Reaction | 55.5 μl |
| RecBCD/ExoV | 1 μl |
| NEB buffer 4 | 1.4 μl |
| ATP | 6.4 μl |

The Master Mix for 100 reactions was as follows, where 8.3 μl was added to each reaction:

TABLE 9

| Reagent | Volume for 50 μl Reaction |
|---|---|
| RecBCD/ExoV | 100 μl |
| NEB buffer 4 | 140 μl |
| ATP | 640 μl |

The digestion reaction was incubated at 37° C. for 1 hour before heat inactivation at 70° C. for 30 minutes, which was cleaned with 6×DNA Clean & Concentrator™-5 columns. About 130 d DNA binding buffer was used, and the columns were eluted in a total volume of 30 μl molecular grade water per cleaning (2×15 μl elutions). The final concentrations were evaluated by Nanodrop Nucleic Acid Quantification (spectrophotometry). The final concentration was about 20 ng/μl to about 35 ng/μl. This stock DNA was frozen after taking an aliquot of pipeline product containing about 300 ng to about 400 ng of DNA for product quality evaluation. Such a large open circular product is inherently fragile and a single freeze/thaw cycle or 24 hours spent at 4° C. can cause significant degradation. This aliquot was frozen separately or used immediately for product quality evaluation while the bulk of the product was safely frozen.

### Example 3: Evaluation of Steps Via Droplet Digital PCR (ddPCR)

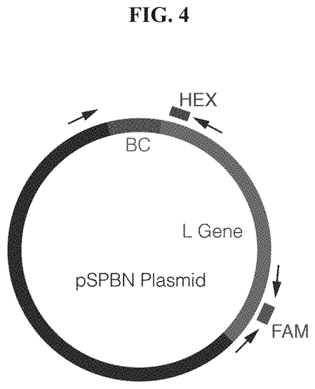

The efficiency of the ligations and exonuclease reactions were evaluated. The HEX probe crossed the ligation site while the FAM probe bound to the L gene, functioning as a control. (Sec. FIG. 4.) The ratio between FAM and HEX amplification was computed in order to determine the percentage of ligated plasmid. The method assumed that all plasmid was intact and that there were no sheared pieces. Accordingly, there would be a large margin of error. The probe or primer sequences used in the evaluation were as follows:

TABLE 10

| | | SEQ |
|---|---|---|
| Probe/Primer | Sequence | ID NO: |
| ddRVBC_tdTom_Forward1 | 5′-CTACTTGTACAGCTCGTCCATG-3′ | 6 |
| ddRVBC_GFP_Forward1 | 5′-CATGGACGAGCTGTACAAGTAAG-3′ | 7 |
| ddRVBC_GFPTdTom_Reverse1 | 5′-CCTCTAACTCGATTGGGTCAATAG-3′ | 8 |
| ddRVBC_L_Forward2 | 5′-TTCGAGGGAACGTTTATCTATC-3′ | 9 |
| ddRVBC_L_Reverse2 | 5′-AATTACAGGCTACCCTTTTGAA-3′ | 10 |
| ddRVBC_GFPTdTom_CN1-HEX | 5′-CCTCTCCAGGATCGAGCATCTTGAAG-3′ | 11 |
| ddRVBC_L_CN2-FAM | 5′-TGGAGTTGGTCCAACGACACCTCA-3′ | 12 |

A stock of probe and primers was prepared as a 20× master mix. Exposure to light was limited as the probes degrade under such exposure.

TABLE 11

| 20× Assay Master Mix | Volume (μl) | [Final μM] |
|---|---|---|
| Forward Primer (100 μm) | 25.2 | 18 |
| Reverse Primer (100 μm) | 25.2 | 18 |
| Probe (100 μm) | 7 | 5 |
| Water | 82.6 | |
| total volume → | 140 | |

The samples that were tested were diluted to 1 ng/μl. From a 1 ng/μl solution, the plasmid was diluted to 1:5000. A PCR master mix was prepared as follows:

TABLE 12

| Reagent | 1× (μl) |
|---|---|
| 2× ddPCR master mix | 12.5 |
| 20× target assay | 1.25 |
| 20× control assay | 1.25 |
| total volume → | 15 |

The master mix (15 μl), water (9 μl), and sample (1 μl) were pipetted into each well. Droplets were made in the droplet generator and PCR was run as follows.

TABLE 13
Cycling conditions: ″DD_PCR_RBC″

| Temp | Time | Cycles |
|---|---|---|
| 95° C. | 10 min | 1 |
| 94° C. | 30 sec | 1 |
| 58° C. | 1 min | 50 |
| 98° C. | 10 min | 1 |
| 8° C. | hold | 1 |

The sample was evaluated on a droplet reader. The total number of FAM+ only droplets, FAM+ and HEX+ droplets, and HEX+ droplets only. The sample ligation ddPCR result was plotted showing the FAM+ only channel (blue) in the upper left quadrant; the HEX+ only (green) in the lower right quadrant; and the double positives (i e, FAM+ and HEX+, orange) in the upper right quadrant (data not shown). The large green to orange ratios signified sheared or slightly degraded DNA. The total positive HEX to total positive FAM was calculated and compared.

TABLE 14

| | | | | | Total | (++)/ |
|---|---|---|---|---|---|---|
| Sample | HEX+ | FAM+ | FAM+ and | Total | HEX+/ | Total |
| Name | Only | Only | HEX+ (++) | Drops | Total FAM+ | HEX+ |
| Ligation | 1203 | 5080 | 914 | 6283 | 0.20 | 0.76 |

### Example 4: AgeI Digest and Agarose Gel Confirmation

An alternate and preferred method of plasmid analysis is gel electrophoresis. The full reactions were run on a gel, and since occasionally, the circularized plasmid can be difficult to see, a restriction enzyme digest assay is often utilized. The RV cDNA plasmid has a single AgeI site. When a ligated plasmid was cut with AgeI, the resulting plasmid was a linear ˜15 kb size piece of DNA. If ligation failed to occur, the plasmid would be split into two fragments of roughly 10 kb and 5 kb, respectively. This size difference was easily resolved on an agarose gel. The relative fractions of the bands, and thus percent ligation of the sample was calculated using ImageJ image processing and analysis software (National Institutes of Health; Bethesda, Md.) The reagents for performing AgeI digest and analysis included: 0.8% agarose gel; TAE buffer; Invitrogen™ SYBR™ Safe DNA Gel stain; CutSmart® Buffer 10×; Template DNA (for controls); Cleaned DNA to evaluate; AgeI-HF (High-Fidelity restriction enzymes with the same specificity as native enzymes, but engineered for significantly reduced star activity and performance in a single buffer (CutSmart® Buffer): NEB); NheI-HF® for controls (NEB): Control Maxi-prepped template; and Gel Loading dye. Purple (6×). no SDS (NEB).

A thin 0.8% agarose gel was poured. i.e., not the low gelling temperature agarose, but regular agarose. For the Owl™ Easy Cast™ B2 Mini Gel Electrophoresis System, a 100 mL gel was poured using the following protocol. Agarose (800 mg) was added to 100 mL of TAE Buffer. The mixture was microwaved for 1.25 minutes and gently swirled in a flask. The mixture was microwaved again for another 45 seconds, swirled, and examined for any undissolved agarose after boiling had ceased. If utilized, the microwaving and swirling steps were repeated. The gel was cooled for 2-3 minutes Invitrogen™ SYBR™ Safe DNA Gel stain was added to the cooled gel (10 μl). The gel was then poured in the cast, avoiding the creation of bubbles. The AgeI reaction was prepared as follows:

TABLE 15

| Component | Volume for 25 μl, 175 ng Reaction |
|---|---|
| DNA to | 175 ng (150 ng also worked if |
| evaluate | concentrations are low) |
| AgeI-HF | 0.5 μl |
| CutSmart | 2.5 μl |
| Buffer | |
| Water | To 25 μl |

The AgeI digest controls used maxi-prepped template. One reaction was treated with only AgeI. Both AgeI-HF and Nhe-HF (0.5 μl NheI-HF per reaction) was reacted with the template to mimic a no ligation condition. The reactions were incubated at 37° C. for 1 hour and heat inactivated at 80° C. for 20 minutes. Each control (5 μl) and each AgeI reaction (10 μl) to be evaluated were loaded into wells on the gel. The template DNA was of extremely high quality and had little to no degradation or shearing. Pipeline DNA often has some residual DNA fragments, which artificially inflates the DNA concentration when evaluated by Nanodrop Nucleic Acid Quantification.

### Example 5: Preserving Diversity while Amplifying: Large Scale Transformations and Plating Maxiprep on Plates

Transformation efficiency experiments were performed on the pipeline product as a proxy for transfection efficacy. Maxi-prepped barcoded pSPBN was transformed at a 10-fold greater rate than the final pipeline product. A protocol (Konermann, et al. Nature, 517(7546):583-588, 2015), which was designed to amplify a library without loss of library diversity was modified and used here. The materials used for transformation included: 8-10 24.5 cm×24.5 cm plates poured with LB agar containing 100 μg/ml ampicillin: 2-8 cm LB agar plates; Invitrogen™ One Shot® OmniMAX™ 2 T1® Chemically Competent E. coli (C854003, ThermoFisher Scientific), or strain with similarly high transformation efficiency; plate spreaders; water bath or heat block; Recovery media which ensures high-efficiency competent cell transformation (80026-1; Lucigen Corporation): and a shaking incubator.

The LB agar plates (24.5 cm×24.5 cm and 8 cm) were placed in a 37° C. incubator. A vial of Lucigen Corporation Recovery media was thawed. The colony forming units (CFU) after post-transformation recovery with the Lucigen Recovery media as opposed to Super Optimal broth with Catabolite repression (SOC) was at least 2-fold higher. While 8 reactions worth of competent cells and DNA were thawed on ice, a water bath or heat block was pre-heated to 42° C. DNA (100 ng) was added to each thawed tube of competent cells and incubated on ice for 30 minutes. The bacteria were heat shocked at 42° C. for 30 seconds without shaking and immediately placed on ice for 2 minutes. Recovery media (1 mL) was added to the vials with the bacteria and gently inverted several times to mix. Recovery media (950 mL) was added to a 14 mL round bottomed culture tube. The mix of recovery media and cells were added to this round bottomed culture tube and shaken at 37° C. for 1 hour. All of the transformations were combined into one tube and 50 Id aliquots of the culture were plated onto the pre-warmed 8 cm plates, while 3 mL aliquots of the culture were plated onto each large 24.5 cm×24.5 cm plate. No visible or movable liquid culture was left on the plates before they were returned to the incubator for incubation overnight at 37° C. The small 8 cm plate was imaged in order to calculate the colony forming units (CFU). LB AMP media was poured onto a plate. A cell spreader was used to scrape the bacteria off of the plate and into the media and pipetted into a pre-weighed centrifuge safe container. This was repeated with an additional 5 mL of media, and the entire process was then repeated for all of the large plates. The bacteria were spun down, supernatant removed, and weight of the tube and pellet recorded. An endotoxin-free maxi prep column was used per 0.5 g to 1 g of pellet.

### Example 6: Packaging Barcoded Rabies Varions

Packaging of barcoded rabies virus genomes of the CVS-N2cΔG (different RV strain; commercially available from Addgene) strain into EnvA-pseudotyped virions (EnvA or EnvB) was performed as described here. A list of materials and equipment is identified in TABLE 16 below, in addition to standard Biosafety Level 2 cell culture laboratory equipment and supplies, as well as additional reagent preparations. An embodiment of the disclosure is directed to a packaging protocol that advantageously scales up the initial transfection step and avoids any amplification step all together, where each amplification step skews the barcode diversity.

TABLE 16

| Material/Equipment | | Cat. # (If | |
|---|---|---|---|
| Name | Source | Applicable) | Additional Comments |
| High diversity barcoded | In-house | N/A | From barcoding and plasmid |
| CVS-N2cΔG vector plasmid | | | preparation. |
| Packaging plasmids: | In-house | N/A | In MaxiPrep quantities. Exact |
| pCAG-N2cN | | | amounts utilized per production |
| pCAG-N2cP | | | run are stated in the Procedures |
| pCAG-N2cL | | | section of this protocol. |
| pCAG-N2cG | | | Plasmids should be dissolved in |
| pCAG-T7pol | | | UltraPure water. |
| HEK-293T/17 cells | ATCC | CRL-11268 | Frozen stocks were generated |
| | | | and stored in-house at low |
| | | | passage number |
| HEK-TVA cells | Salk Institute | N/A | Frozen stocks were generated |
| | Viral Core | | and stored in-house at low |
| | | | passage number |
| Neuro2a-EnvA cells | Columbia | N/A | Frozen stocks were generated |
| | University | | and stored in-house at low |
| | Zuckerman | | passage number |
| | Virus Core | | |
| Antibiotic-antimycotic | ThermoFisher | 15240-062 | — |
| | Scientific | | |
| Fetal bovine serum, | ThermoFisher | 10082147 | — |
| certified, heat-inactivated | Scientific | | |
| DMEM with GlutaMAX | ThermoFisher | 10569010 | A suitable alternative was to |
| supplement, pyruvate, high | Scientific | | use DMEM with GlutaMAX, |
| glucose | | | HEPES, and high glucose, |
| | | | supplementing it with sodium |
| | | | pyruvate before use. |
| DPBS, no calcium, no | ThermoFisher | 14190-144 | — |
| magnesium | Scientific | | |
| DPBS, with calcium & | ThermoFisher | 14040-133 | — |
| magnesium | Scientific | | |
| 0.05% Trypsin EDTA with | ThermoFisher | 25300-054 | — |
| phenol red | Scientific | | |
| Opti-MEM reduced serum | ThermoFisher | 51985-034 | — |
| media, with GlutaMAX | Scientific | | |
| supplement | | | |
| Sucrose | Millipore | S1888 | — |
| | Sigma | | |
| Poly-L-Lysine solution, | Millipore | P4707 | Poly-D-Lysine was a suitable |
| 0.01%, sterile filtered | Sigma | | alternative |
| Benzonase nuclease, 99% | Millipore | 70664 | — |
| purity, 10 kUnits | Sigma | | |
| Xfect transfection reagent | Clontech | 631318 | — |
| 38.5 mL ultra-clear thin- | Beckman | 344058 | — |
| wall ultracentrifuge tubes | Coulter | | |
| Ultracentrifuge with | Beckman | — | Or equivalent centrifuge & |
| SW32Ti rotor | Coulter | | rotor |
| T-225 tissue culture treated | Falcon | 14-826-80 | — |
| flasks | | | |
| 15 cm tissue culture treated | Corning | 08-772-24 | — |
| culture dishes | | | |
| 0.22 μm PES filters, | — | — | — |
| syringe-driven filter tips | | | |
| 0.22 μm PES filters, | — | — | — |
| vacuum-driven 500 mL | | | |
| rapid flow with bottle | | | |
| 1.5 mL low protein binding | — | — | — |
| microtubes | | | |
| 15 mL conical | — | — | — |
| polypropylene centrifuge | | | |
| tubes, sterile | | | |
| 20 mL sterile syringes with | — | — | — |
| Luer-Lok tips | | | |
| Benchtop vortex | — | — | — |

TABLE 17
Recovery of Barcoded Virions from cDNA

| Reagent/Material | Steps |
|---|---|
| Coated T-225 flasks | T-225 flasks were coated with enough poly-L-lysine |
| | solution to cover the entire bottom surface |
| | (approximately 18 mL). |
| | The flasks were incubated overnight at 37° C. |
| | The poly-L-lysine solution was aspirated away. |
| | 25 mL of sterile water was used to rinse twice. |
| | The flask was dried under sterile conditions and the |
| | caps sealed with Parafilm for long-term storage at |
| | 4° C. |
| | Before use, the flasks were rinsed with DPBS |
| | (−Ca, −Mg) was used to rinse. |
| 10% FBS media | The following were added to DMEM + GlutaMAX |
| | (volumes for 500 mL): |
| | 1:100 antibiotic-antimycotic (5 mL) |
| | 10% fetal bovine serum (50 mL) |
| | If using DMEM + GlutaMAX with HEPES, sodium |
| | pyruvate was added before use. |
| 10% FBS media | The following were added to DMEM + GlutaMAX |
| | (volumes for 500 mL): |
| | 1:100 antibiotic-antimycotic (5 mL) |
| | 10% fetal bovine serum (50 mL) |
| | If using DMEM + GlutaMAX with HEPES, sodium |
| | pyruvate was added before use. |
| 20% sucrose in | For 20 mL, 4 g of sucrose was dissolved in 15 mL |
| DPBS cushion | DPBS (−Ca, −Mg). |
| (for ultracentrifugation) | The final volume was topped off to 20 mL with |
| | DPBS (−Ca, −Mg). |
| | The solution was filtered through a 0.22 μm PES |
| | syringe-driven filter. |
| | The solution was stored at 4° C. |

HEK-293T/17 cells were plated in coated T-225 flasks, with a minimum of 2 flasks and a maximum of 6 flasks depending on the intended batch size. Cells were plated at sufficient density to reach 85-95% confluency 1-2 days after the flasks were seeded. Cells were seeded at least 24 hours prior to transfection. On the day of transfection, the Xfect transfection reagents were thawed and vortexed well after they thawed to room temperature. A tube containing the Xfect buffer was prepared, scaling the amount shown in the below table by the number of T-255 flasks or equivalent growth surface area. The polymer was not added at this step.

TABLE 18
Transfection reagent volumes (per T225 flask)

| | Amount | |
|---|---|---|
| Component | (μL) | Comments |
| Xfect buffer | 2250 | — |
| Xfect polymer | 84.375 | Was added to the plasmid |
| | | mixture in the below step |

The packaging plasmid mixture was prepared in a 15 mL conical tube containing Xfect buffer based on the values shown below. The amounts were scaled according to the total number of T225 flasks or equivalent growth surface area (final DNA concentration of the total mixture was 1.25 μg/cm²) and adjusted by 1.1× for pipetting errors.

TABLE 19
Packaging plasmid mixture (per T-225 flask)

| Component | Molar ratio | 1.1× Amount (μg) |
|---|---|---|
| RbV Vector | 4 | 131.36 |
| pCAG-N2cN | 4 | 52.11 |
| pCAG-N2cP | 2.5 | 30.15 |
| pCAG-N2cL | 1 | 23.70 |
| pCAG-N2cG | 1.5 | 20.26 |
| pCAG-T7pol | 1.5 | 23.66 |
| Total | — | 281.24 |

The Xfect polymer was added to the plasmid mixture and scaled according to the values in the Transfection reagent volumes table. No contact was made between the pipette tip and the plasmid mixture. The contents were allowed to sit at room temperature (20° C.-25° C.) for 30 seconds. The transfection mixture was vortexed on high for 10 seconds, and the contents were briefly spun down. The mixture was incubated at room temperature for 10 minutes such that complexes formed. The media in the T-225 flasks containing the HEK-293T/17 cells was aspirated away and replaced with 20 mL warm Opti-MEM. After the 10 minute incubation time, the transfection mixture was added to the flask drop-wise using a P1000 micropipette, distributing the drops evenly around the flask. Direct contact between the transfection mixture and the flask walls was avoided. The flask was gently agitated to mix and distribute the transfection mixture and then incubated for 5 hours in the incubator at 35° C., 5% CO₂.

After the 5 hour incubation, the media in the transfected T-225 flasks was replaced with fresh 10% FBS media and incubated at 35° C., 5% CO₂. At 1 day post-transfection (d.p.t.), 15-20 mL of fresh 5% FBS media was added to each T-225 flask. At 2 d.p.t., the media was replaced in each T-225 flask with 30 mL of 5% FBS media. At 3 d.p.t., 15-20 mL of fresh 5% FBS media was added to each T-225 flask. At 4 d.p.t., the media in each T-225 flask was replaced with 30 mL of 5% FBS media. Fluorescent clusters should be visible on a suitable inverted fluorescence microscope by this timepoint. Fluorescent clusters of cells generating barcoded rabies virus were shown in a fluorescence and brightfield composite image of HEK-293T/17 cells transfected with packaging plasmids and barcoded rabies virus genome plasmids that contain the red fluorescent marker gene tdTomato. Images were taken 4 days post-transfection. Cells that were generating rescued rabies virions had high red fluorescence due to the expression of tdTomato from the rescued barcoded rabies virus genome. Cells adjacent to the virion-generating cells were secondarily infected by virions budding off from the virion-generating cell, resulting in clusters of cells with red fluorescence that were typically observed by this timepoint (Figure not shown.) At 4 d.p.t. or earlier, there were Neuro2A-EnvA cells seeded into 15 cm dishes (1 dish per transfected T-225 flask) that reached 85-95% confluency by 6 d.p.t. for pseudotyping. At 5 d.p.t., 15-20 mL of fresh 5% FBS media was added to each T-225 flask.

### Example 7: Pseudotyping with EnvA Glycoprotein Coat

If an unpseudotyped virus was desired, this step was skipped and continued at virion collection. Otherwise, Pseudotyping Stage 1 at 6 d.p.t. was performed as follows. The media from the 15 cm dishes of Neuro2A-EnvA cells was aspirated away and 20 mL of fresh 5% FBS media was added. The media was collected and filtered from the transfected T-225 flasks. The virion-containing media was filtered from up to 2×T-225 flasks through a single 0.22 μm PES 500 mL vacuum-driven filter. The Neuro2A-EnvA cells were infected by adding the filtered virion-containing media, dividing it equally between all of the 15 cm dishes of Neuro2A-EnvA cells. The Neuro2A-EnvA cells were incubated at 35° C., 5% CO₂ for approximately 6 hours for infection. After the 6-hour incubation, the media was aspirated away and the dishes rinsed twice with cold DPBS (+Ca, +Mg) and gently pipetted to ensure that cells do not detach. Trypsin-EDTA (5 mL) was added and incubated at 35° C. for 30 seconds. A P1000 pipette was used to mechanically dissociate the cells from the dish. FBS media, 10% (20 mL) was added to each dish and all of the contents were transferred to a sterile 50 mL tube (I tube per 15 cm dish). Another 10 mL of 10% FBS media was added to each dish for rinsing and any remaining cells were collected. This volume was added to the contents of the corresponding 50 mL tube. The cells were pelleted using a centrifuge (300 g, 4 min). The supernatant was aspirated and the cells were resuspended in 25 mL DPBS (−Ca, −Mg). The cells were pelleted using a centrifuge (300 g, 4 min). The supernatant was aspirated and the cells were resuspended in 10 mL of 5% FBS media. The cells were replated in new 15 cm dishes labeled “P1” (“Pseudotyping 1”). FBS media, 5% (10 mL) was added to a final volume of 20 mL per dish and incubated at 35° C., 5% CO₂.

At 7 d.p.t.: Pseudotyping Stage 2 occurred using the below steps. The media was aspirated away in the P1 dishes. Trypsin-EDTA (5 mL) was added and incubated at 35° C. for 30 seconds. A P1000 pipette was used to mechanically dissociate the cells from the dish. FBS media, 10% (20 mL) was added to each dish and all of the contents were transferred to a sterile 50 mL tube (1 tube per 15 cm dish). Another 10 mL of 10% FBS media was added to each dish to rinse and any remaining cells were collected. This volume was added to the contents of the corresponding 50 mL tube. The cells were pelleted using a centrifuge (300 g, 4 min). The supernatant was aspirated away and the cells were resuspended in 25 mL DPBS (−Ca, −Mg). The cells were pelleted using a centrifuge (300 g, 4 min). The supernatant was aspirated away and the cells were resuspended in 10 mL of 5% FBS media. The cells were replated in new T-225 flasks labeled “P2” (“Pseudotyping 2”). FBS media, 5% (20 mL) was added to a final volume of 30 mL per flask and incubated at 35° C., 5% CO₂. From 8 d.p.t. until collection at 11 d.p.t or 12 d.p.t., 3-5 mL of fresh 5% FBS media was added to each flask daily. The recommended volume of 45 mL per flask was not exceeded.

### Example 8: Virion Collection

Before virion collection, the fluorescence in the P2 flasks was checked. Empirically, production of EnvA-pseudotyped virions peaks approximately 3-4 days after initial infection, and most of the Neuro2A-EnvA cells should be fluorescently labeled 1-2 days after Pseudotyping Stage 2. An EGFP marker was introduced into the Neuro2A-EnvA cells when the cell line was generated. Supernatant from the flasks was collected and the media was pooled into a suitably large bottle (e.g., 500 mL Nalgene® bottle), noting the total volume collected. Benzonase nuclease was added at 1:1000 of the total supernatant volume and incubated for 30 minutes at 37° C. The rotor and supernatant were chilled to 4° C. The supernatant was incubated for 30 minutes and filtered using 0.22 μm polyethersulfone (PES) vacuum-driven filters. The volume was divided across several filters (at most 150 mL per 500 mL filter to avoid clogging the filters), and the filtered media was collected into a single bottle. The ultracentrifuge tubes were inserted into the rotor buckets and 2 mL of 20% (w/v) sucrose in DPBS (−Ca, −Mg) was added. The filtered supernatant was divided equally between the centrifuge tubes, adding a maximum of 33 mL. The total volume per tube did not exceed 35 mL. DPBS was added to top up and balance the tubes as needed ensuring that no tubes were left empty. The ultracentrifuge was loaded and run at 20,000 RPM (for SW32Ti rotor) for 2 hours at 4° C. with maximum acceleration and braking. The tubes were unloaded and decanted to discard the supernatant while keeping the tube inverted. The pellet containing the virions was not disrupted. The inverted tubes were placed on a sterile cloth or paper towels were used to wick away excess media. Residual media inside the tube was aspirated away within 1 inch of the tube mouth. DPBS (−Ca, −Mg) (15 μL) was added to each tube and placed on ice. The tubes were sealed with Parafilm® and placed on an orbital shaker at 4° C. for 8 hours. The virus was resuspended by gently pipetting around the base of the tube and the suspension was pooled into a 1.5 mL low protein binding tube. Aliquots were made in low protein binding tubes and stored at −80° C. Repeated freeze/thaw cycles were avoided after freezing aliquots as this would reduce virus titer.

### Example 9: Quality Control and Titration

A 12-well plate was seeded with HEK-TVA cells and a final volume of 1 mL 10% FBS media was added, and a separate plate of HEK-293T/17 cells was also seeded. The plates were incubated at 37° C., 5% CO₂. When the cells were at approximately 80% confluent, a test aliquot of the virus was thawed on ice. A serial dilution was performed using DPBS (−Ca, −Mg) in separate sample tubes (1e-1, 1e-2, 1e-3, 1e-4, 1e-5) starting from the 1× test aliquot. For each dilution (1× to 1e-5), a corresponding well in the 12-well plate of HEK-TVA cells was labeled and 1 μL of the sample was added at the matching dilution. The plate was agitated to mix well and incubatee at 37° C., 5% CO₂. On the same day, 1 μL of the test aliquot was added to a well in the 12-well plate of HEK-293T/17 cells marked “control”. The plate was agitated to mix well and incubated at 37° C. 5% CO₂. After infection for 24 hours, the wells were checked for fluorescence. There should be no fluorescent cells in the HEK-293T/17 “control” well if the pseudotyping procedures were performed well, with a tolerance of at most 2 fluorescent cells in the entire “control” well. The well of HEK-TVA cells was infected with 1 μL of virus sample at 1× dilution and had approximately 80% of the cells fluorescently labeled by the virus. The wells from 1e-1 to 1e-5 were examined and a well with approximately 5-10 cells per field-of-view under a 10× magnification objective was identified. The number of fluorescent cells was counted to obtain an estimate for the total number of infected cells in the well, and the dilution factor was scaled to obtain the virus titer in infectious units per mL (IU/mL). For example, an average of 8.5 cells was obtained after counting 30 regions in the well when counting from 1e-4 well with a field of view that is 1/100 (1e-2) the area of the full well. Since 1e-3 mL was added to the well, the estimated titer of the virus batch when scaled for dilution was calculated as (8.5/1e-2/1e-4/1e-3)=8.5×10⁹ IU/mL. A test sample for sequencing analysis was prepared to determine the barcode diversity.

The following table presents solutions and recommended actions for any issues identified in the methods described here.

TABLE 20

| | Possible Explanation(s)/Recommended |
|---|---|
| Observed Problem | Action(s) |
| Precipitation of plasmid DNA when | Precipitation has been noted to occur when the |
| added to the Xfect buffer. | plasmid DNA was dissolved in TE. |
| | Ensure that the plasmids are dissolved in UltraPure |
| | water and repeat the DNA MaxiPrep if necessary. |
| | Ensure that the Xfect buffer is brought to room |
| | temperature before use. |
| DNA appears to be precipitating out | Ensure that the micropipette tip containing the Xfect |
| and sticking to the micropipette tip. | polymer does not touch the Xfect buffer containing |
| | the plasmid DNA mixture. |
| Flask contains many detached cells. | Ensure that the T-225 flasks are coated with |
| | PLL/PDL, and rinsed with DPBS (−Ca, −Mg) before |
| | seeding the cells. |
| Extensive/excessive cell death after | Try gradually reducing the total DNA concentration |
| transfection, with most cells in the | down from 1.25 μg/cm² to lower values. Note that |
| flask detached. | this may lead to lower diversity virion libraries. |
| No fluorescence is observed. | Ensure that all the plasmids are added in the correct |
| | ratios, and that the barcoded rabies vector plasmid |
| | contains a fluorescent marker that is compatible with |
| | the microscope being used. |
| Unable to check fluorescence | If the T-225 flask is not compatible with |
| because the T-225 flask is too | microscopes available, 1 mL media samples can also |
| large/incompatible with the | be taken from the transfected T-225 flasks, then |
| fluorescence microscope. | filtered and added to a HEK-293T/17 cells grown in |
| | a separate 12-well plate. Check fluorescence in the |
| | test well at least 24-hours after the test infection. |
| Neuro2A-EnvA cells are detaching | Use coated 15 cm dishes and T-225 flasks for |
| during washing steps. | Neuro2A-EnvA cells, and ensure that washing steps |
| | are done using cold DPBS (+Ca, +Mg). Cells can |
| | detach easily if dishes are washed with DPBS (−Ca, −Mg). |
| Media color is very yellow before | Try using DMEM + GlutaMAX with HEPES to |
| virus collection. | increase buffering capacity. |
| Virus suspension is very viscous | Benzonase nuclease was added to reduce viscosity |
| when handling, | of the final suspensions, and 0.22 μm filters are used |
| | to reduce the amount of cell debris that can also add |
| | to viscosity or handling issues (e.g. clogging during |
| | virus injections). |
| | When aliquoting, debris may still be observed |
| | settling to the bottom of the 1.5 mL collection tube. |
| | Avoid distributing this debris to the aliquots by |
| | pipetting off from the top and discard the debris. |
| High “leak” of unpseudotyped | Unpseudotyped virions leaking into the final |
| virions in pseudotyped virus batch; | suspension are likely to be a result of inadequate |
| many fluorescently-labeled cells in | trypsinization and washing during the pseudotyping |
| the “control” HEK-293T/17 well. | steps, or contamination of Neuro2A-EnvA cell |
| | stocks with Neuro2A-N2cG cells that will generate |
| | unpseudotyped virions. |
| Low titer (<1 × 10⁷ IU/mL). | Low titers can be caused by errors in concentration |
| | by ultracentrifugation, e.g. leaving the pellet and |
| | supernatant in the tubes for too long after the |
| | ultracentrifuge has stopped. Ensure that the tubes are |
| | removed promptly after ultracentrifugation is |
| | complete, and that the supernatant is gently decanted |
| | out of the tube. |
| | Low titers may also be caused by issues with the cell |
| | lines, such as contamination or loss of transgene |
| | expression in the case of the Neuro2A-EnvA cells. |
| | Check the Neuro2A-EnvA cells for expression of the |
| | EGFP fluorescence marker. |

### Example 10: Sequencing Genomic Barcodes from Bulk Barcoded Rabies Virus (RV)

Rabies Virus RNA genomes were extracted using the ZR Viral DNA/RNA Kit (Zynogen, D7020) from 1) high-titer aliquots of final, ultra-centrifuged libraries (5 μl˜10⁶ RV genomes) or 2) following PEG-based particle precipitation (Abcam, ab 102538) from cell culture media (1 ml media, resuspended in 100 μl re-suspension solution) and eluted in 15 μl of RNAase free water (Zymogen). Extracted RNA was quantified using a high-sensitivity RNA ScreenTape (Agilent, 2200 TapeStation). To count genomic barcode sequences, a DNA oligonucleotide (UMI-pSPBNg_GFP_F2) carrying a 12 base pair unique molecular identifier (UMI) was first hybridized to a negative stranded RNA adjacent to the genomic barcode region and reverse-transcribed using 5× Maxima Reverse Transcriptase (Thermo Fisher Scientific, EP0753). RNA present in RNA/DNA duplexes digested with Ribonuclease H (RNAase H: New England Biolabs, M0297S) and UMI-tagged single-stranded DNA (ssDNA) copies of RV genomes were cleaned with Agencourt AMPure XP beads (1:1, Beckman Coulter, A63881), resuspended in 25 μl H₂O, and concentration quantified (NanoDrop™ 2000, ThermoFisher Scientific). To prepare UM-tagged genomic barcodes for Illumina sequencing, PCR (50 ng ssDNA, 20-27 cycles; Kappa HiFi HotStart ReadyMix 2×. Kapa Biosystems, KM2602) was performed using a forward primer (P5-TSO_Hybrid) carrying the P5 site and complementary to a unique handle on the hybridization oligonucleotide (UMI-pSPBNg_GFP_F2). The reverse primer (P7il-L5UTR_Hybrid_v2) carrying the P7 site flanks the genomic barcode, hybridizing to the 5′ UTR of the L gene. The 261 bp amplicon was cleaned and size-selected using Agencourt AMPure XP beads (0.6:1 retaining supernatant, followed by 1:1), resuspended in 10 μl H₂O, and concentration quantified using the 2100 Bioanalyzer (Agilent) with the High-Sensitivity DNA assay (Agilent, 5067-4626). UMI-tagged genomic barcode libraries (20 μM) were then sequenced on the Illumina NextSeq® platform, following standard Illumina library preparation guidelines. A custom R1 primer (Read1CustomSeqB) was used to initiate 110 Read 1 cycles, covering 1) the UMI (bps 1:12), fixed sequence at the 3′ end of the GFP gene (bps 13: 49) and the barcode containing cassette (bps 50: 110).

### Example 11: Sequencing Barcoded Rabies Virus Transcripts from Single-Cell RNA-Seq

To prepare Rabies Virus (RV) viral barcode libraries for sequencing, barcode-carrying RV GFP transcripts were selectively PCR amplified from cDNA following single-cell RNA-seq library generation (Chromium Single Cell 3′ Library & Gel Bead Kit v3). P7-containing forward primers targeting the 3′ region of the green fluorescent protein (GFP) transcript (BC_Seq_P7il_GFP_v4c) were coupled with P5-containing reverse primer (P5-10×_Hybrid) complementary to the PCR handle (equivalent in sequence to the Illumina R1 primer site) introduced by 10×iGEM beads. Between 12-18 cycles of PCR reactions were run. The resulting amplicons were cleaned by AMPure XP beads (0.6:1 retaining supernatant, followed by 1:1), quantified using the 2100 Bioanalyzer (Agilent), and prepared for Illumina sequencing following Illumina's guidelines. Single-cell barcoded RV transcript libraries (1.8 μM, 20% PhiX) were multiplexed on Illumina NextSeq® 550, generating between 38-184 million reads per library. The 10× cell barcode (bps 1:16) and UMI (bps 17-26) were captured by 26 cycles on Read 1; 98 cycles on Read 2 sequenced through the fixed 3′ GFP sequence (1-28 bps) and into the barcode cassette (bps 29-98).

### Example 12: Informatic Extraction and Correction of Rabies Virus Barcodes

To extract Rabies Virus (RV) barcodes from Illumina sequence data, custom software was written to identify the barcode cassettes based on alignments to fixed, flanking sequences. Extracted barcode sequences were filtered based on Illumina quality scores (all bases >10 Phred Quality) and length (n=2 10 bp sequences).

To collapse barcode mutations (induced through PCR and sequencing errors in addition to errors caused by RV replication/transcription), two barcode collapse algorithms were developed and deployed on either the 1) genomic barcodes in hyper-diverse RV libraries (sequence space >500K barcodes) or 2) transcript barcode present in single-cells (sequence space <10 barcodes).

For RV libraries carrying hyper-diverse genomic barcodes, “mutation path collapse” (MPC) was performed. MPC started with the most abundant (“core”) barcode in the library and searched that vast barcode sequence space (>500K barcodes) for other barcodes that are hamming edit-distance (ED) n:=1 away (“ED1 neighbors”). ED1 neighbors were collected and the MPC search was repeated, collecting a second set of ED1 neighbors MPC process continued on each new set of ED1 neighbors until no additional sequences in the library were identified. The UMIs from the full set of collected barcodes were added to the counts for the “core” barcode and the barcodes belonging to the ED1 neighbor collection were removed from the library. By following an edit-distance path. MPC collapses mutant variants of “core” barcodes while avoiding spurious collapse of true “core” barcodes to each other.

For single-cell RNA-seq datasets, where RV barcodes are detected on Green Fluorescent Protein (GFP) transcripts, “adaptive edit distance (AED) collapse” was performed. The AED algorithm calculated hamming edit-distances for all barcodes detected in single cells. For the first barcode detected (a “test” barcode), pairwise ED relationships were calculated across all other barcodes in the cell. The resulting distribution was plotted in a histogram with a total number of bins corresponding to all possible ED values (1 through 20). The AED algorithm then attempted to identify the smallest ED bin with 0 measurements. Barcodes in bins smaller than this “trough” were collapsed into the “test” barcode and removed from the cell. The process was repeated for all remaining barcodes. If no ED bin with 0 measurements was identified, the algorithm defaulted to collapsing all barcodes with hamming-edit distance <10. Importantly. AED happened upstream of UMI-based counting: thus, collapsed barcodes with the same UMI were not counted multiple times. AED leveraged the assumption of a small barcode sequence space within single cells to collapse related barcodes initially distinguished by mutations created through viral transcription, PCR amplification, or sequencing errors.

### Example 13: Inference of Synaptic Networks Containing Identified “Starter” Cells in Mouse Cortical Cultures Via RV Barcode Sharing

Mouse cortical culture were transduced with high concentrations of Cre-dependent adeno-associated viruses (AAVs) expressing a TVA receptor mCherry fusion protein (TCB) as well as RV glycoprotein (G). The number of starter cells in each well was controlled by concentration of a 3rd AAV expressing Cre under the synapsin promoter. Low concentrations of Cre-expressing AAV induce a sparse number of starter cells.

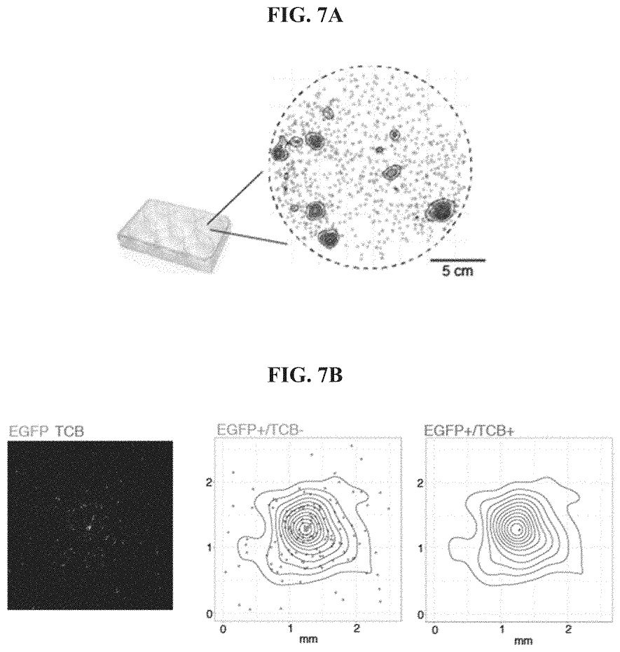

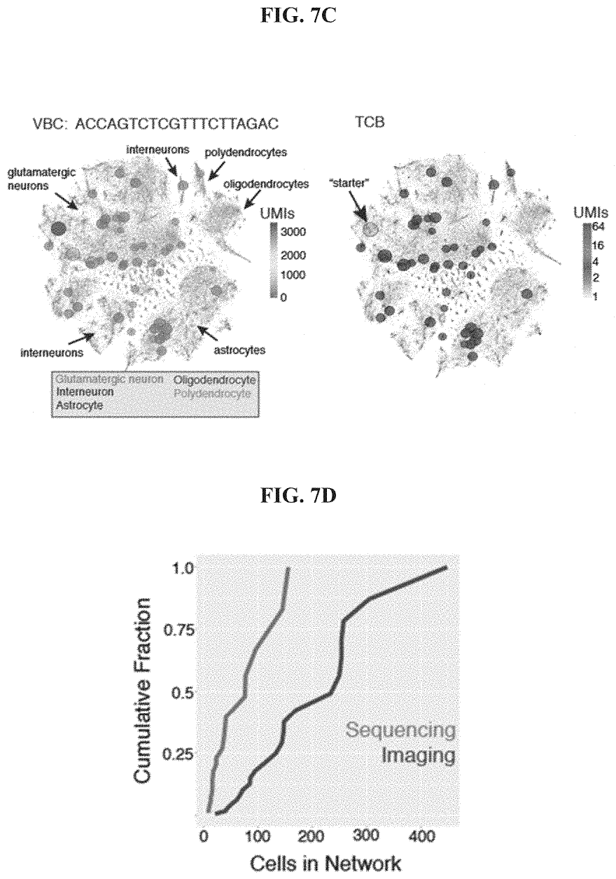

Cortical cultures were grown on glass coverslips, fixed and imaged. Contour lines demarcate areas of high EGFP cell density. Each green dot corresponds to a EGFP+ (RV infected) cell EGFP+/TCB+ (“starter”) cells are shown in magenta. For example, these cells may be found in the large cell density found in the lower right, in the smallest and largest cell density areas in the center, and in the central cell density of the upper left quandrant of cortical cultures. Areas of high EGFP+/TCB− cell density surrounding EGFP+/TCB+ starter cells are presumed to be presynaptic network originating from the starter cell in the immediate vicinity. EGFP+/TCB− cells distant from starter cells are in presynaptic networks but imaging alone cannot identify their starter cell of origin. (See, FIG. 7A). A magnified view of a single sparse network enabled the finding that EGFP+/TCB− cells are presumed to be part of the presynaptic network innervating the EGFP+/TCB+ starter cell. (See. FIG. 7B). A single cell expressed TVA receptor mCherry fusion protein (TCB) transcripts, and thus is presumed to be the starter cell. For this example, a RV particle carrying the viral barcode (VBC) sequence. “ACCAGTCTCGTTTCTTAGAC” (SEQ ID NO. 2) infected a glutamatergic neuron in which adeno-associated viruses (AAVs) drove the expression of TCB and the RV Glycoprotein (G). This VBC carrying particle then replicated and spread from the dendrites of the starter cell into presynaptic cells lacking TCB/G. These presynaptic cells included glutamatergic neurons, interneurons, and non-neurons. (See, FIG. 7C).

### Example 14: Using SBARRO to Identify Directional, Cell Type-Specific Synaptic Networks

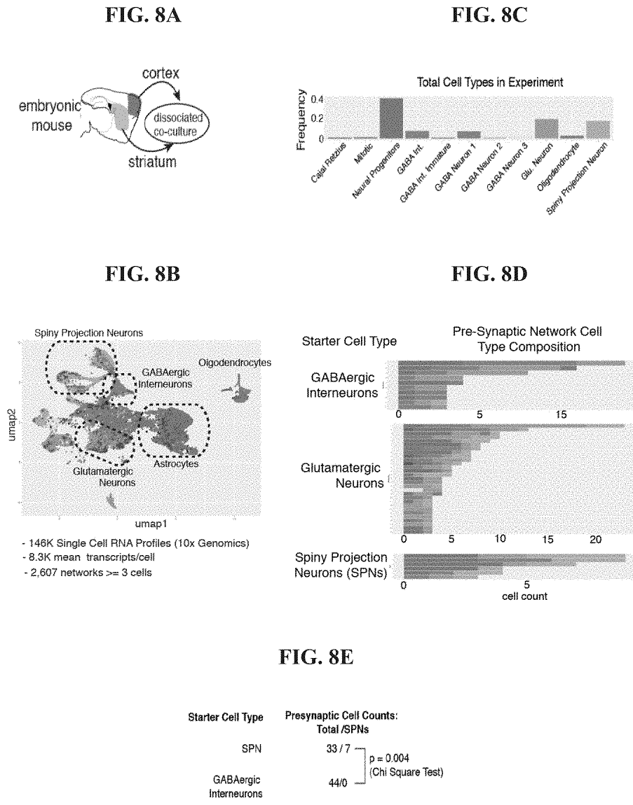

Synaptic networks were cultured from dissociated cells originating from two different developing brain regions (cortex and striatum), each of which contain distinct cell types. (See, e.g., FIG. 8). The cortex and striatum of aged E16 embryonic mice were dissociated into single cells and co-cultured for 14 days before SBARRO infection. The molecular identities of 146,000 single-cell RNA profiles from 22 SBARRO libraries were determined. Endogenous gene expression patterns were used to determine the major cell types, including spiny projection neurons, GABAergic interneurons, astrocytes, and glutamatergic neurons (see. FIG. 8B). These data yielded a mean of 8,300 (8.3K) transcripts/cell and resulted in the identification of n=2,607 independent putative synaptic networks with >=3 cells. Individual pre-synaptic network cell type cell counts originating from starter cell types were identified as including subsets of GABAergic interneurons, glutamatergic neurons, and spiny projection neurons (SPNs) (FIG. 8D). The frequencies of different cell types were determined, where neural progenitors, glutamatergic neurons, and SPNs had the highest frequencies. (See. FIG. 8C). It was found that SPNs tend to innervate other SPNs, but not GABAergic interneurons, following in vivo predictions from electrophysiological and anatomical analyses of mouse striatal cell type connectivity. (See, FIG. 8E).

As various changes can be made in the above-described subject matter without departing from the scope and spirit of the present disclosure, it is intended that all subject matter contained in the above description, or defined in the appended claims, be interpreted as descriptive and illustrative of the present disclosure. Many modifications and variations of the present disclosure are possible in light of the above teachings. Accordingly, the present description is intended to embrace all such alternatives, modifications and variances which fall within the scope of the appended claims.

### Other Embodiments

As various changes can be made in the above-described subject matter without departing from the scope and spirit of the present disclosure, it is intended that all subject matter contained in the above description, or defined in the appended claims, be interpreted as descriptive and illustrative of the present disclosure. Many modifications and variations of the present disclosure are possible in light of the above teachings. Accordingly, the present description is intended to embrace all such alternatives, modifications and variances which fall within the scope of the appended claims.

The recitation of a listing of elements in any definition of a variable herein includes definitions of that variable as any single element or combination (or subcombination) of listed elements. The recitation of an embodiment herein includes that embodiment as any single embodiment or in combination with any other embodiments or portions thereof.

All patents and publications mentioned in this specification are herein incorporated by reference to the same extent as if each independent patent and publication was specifically and individually indicated to be incorporated by reference.

What is claimed is:

1. A rabies virus genome, comprising: a 3′ to 5′ linear, nucleic acid sequence encoding a rabies virus (RV) nucleoprotein, a RV phosphoprotein, a RV matrix protein, a barcode, and a RV polymerase.

2. The rabies virus genome of claim 1, wherein the one or more nucleic acid sequence encoding the RV nucleoprotein, the RV phosphoprotein, the RV matrix protein, the barcode, or the RV polymerase is an endogenous gene or transgene.

3. The rabies virus genome of claim 1, wherein the barcode comprises a restriction enzyme cassette.

4. The rabies virus genome of claim 1, further comprising a selectable marker or detectable moiety.

5. The rabies virus genome of claim 4, wherein the detectable moiety is a fluorophore.

6. The rabies virus genome of claim 3, wherein the restriction enzyme cassette divides the barcode into two halves.

7. A viral genome, comprising: a nucleic acid sequence encoding some viral proteins of a viral species and a barcode, wherein said viral genome is in a viral particle of the viral species that infects through synaptic junctions.

8. The viral genome of claim 7, wherein the nucleic acid sequence encodes all viral proteins of the viral species.

9. The viral genome of claim 6, wherein the barcode comprises a restriction enzyme cassette.

10. The viral genome of claim 9, further comprising a selectable marker or detectable moiety.

11. A rabies virus particle comprising the rabies virus genome of claim 1.

12. A viral particle comprising the viral genome of claim 9.

13. A polynucleotide encoding a barcode, comprising a restriction enzyme cassette, wherein the restriction enzyme cassette separates the barcode into two equal or unequal halves.

14. A method of constructing a hyper-diverse barcoded plasmid library, comprising:
a) amplifying a template plasmid in an amplification reaction using a forward primer and a reverse primer, wherein the forward primer and the reverse primer each comprise from 5′ to 3′ a complementary region, a barcode, a linker, and a restriction enzyme site, wherein the restriction enzyme sites of the forward primer and the reverse primer generate 3′ compatible overhangs when cleaved;
b) generating double-stranded linear amplicons, each comprising the template plasmid sequence comprising barcodes at the termini of the amplicons;
c) digesting the amplicon with a restriction enzyme to produce a digested product comprising 5′ and 3′ overhangs;
d) ligating the digested product to produce a circular barcoded plasmid; and
e) selectively digesting linear DNA with an exonuclease.

15. The method of claim 14, wherein the exonuclease is RecBCD.

16. The method of claim 14, wherein the barcode sequence is separated from the restriction enzyme site by 3 base pairs to 25 base pairs; the linker sequence is 3 base pairs to 30 base pairs in length; and/or the complementary region is 18 base pairs to 200 base pairs in length.

17. A library of hyper-diverse barcoded plasmids, wherein the hyper-diverse barcoded plasmid is a circular plasmid and comprises at least two identifiable barcode sequences separated from a restriction enzyme site by a linker sequence.

18. A method of inferring synaptic connectivity, comprising:
a) contacting one or more starter cells susceptible to infection with a library of articles each comprising a rabies virus genome of claim 9;
b) replicating the virus particle within the one or more infected starter cells;
c) allowing the virus to infect one or more additional cells, each synaptically connected to the starter cell;
d) optionally sorting the cells to select the rabies virus infected cells;
e) creating single-cell RNA-seq libraries from the infected cells;
f) high-throughput sequencing of the single-cell RNA-seq libraries to identify RNA sequences and viral barcodes present within each cell; and
g) inferring synaptic connectivity of cells based on the sharing of at least one identifiable barcode, wherein cells that share an identifiable barcode are inferred to be in the same synaptic network.

19. The method of claim 18, further comprising: identifying cell types from the identified RNA sequences.

20. The method of claim 18, wherein the identifiable barcode comprises a selectable marker or detectable moiety.

21. The method of claim 18, where the selectable marker or detectable moiety is a fluorophore, an antibody resistance cassette, a capture molecule, a biotin molecule, streptavidin molecule, or an antigen.


SEQUENCE LISTING (13 sequences)

SEQ ID NO: 1 - DNA, 16 bp, Rabies virus
ccgtggagtaccatca

SEQ ID NO: 2 - DNA, 20 bp, Rabies virus
accagtctcgtttcttagac

SEQ ID NO: 3 - DNA, 67 bp, Artificial Sequence
  Description of Artificial Sequence Synthetic oligonucleotide
  modified_base (18)..(27): a, c, t, g, unknown or other
acctgtggcgccactgcnnnnnnnnnncttttcagtcgagaaaaaaacattagatcagaagaacaac

SEQ ID NO: 4 - DNA, 65 bp, Artificial Sequence
  Description of Artificial Sequence Synthetic oligonucleotide
  modified_base (18)..(27): a, c, t, g, unknown or other
acctgtggcgccaccggnnnnnnnnnntagcctacttgtacagctcgtccatgccgtacaggaac

SEQ ID NO: 5 - DNA, 65 bp, Artificial Sequence
  Description of Artificial Sequence Synthetic oligonucleotide
  modified_base (18)..(27): a, c, t, g, unknown or other
acctgtggcgccaccggnnnnnnnnnntagcttacttgtacagctcgtccatgccgagagtgatc

SEQ ID NO: 6 - DNA, 22 bp, Artificial Sequence
  Description of Artificial Sequence Synthetic oligonucleotide
ctacttgtacagctcgtccatg

SEQ ID NO: 7 - DNA, 23 bp, Artificial Sequence
  Description of Artificial Sequence Synthetic oligonucleotide
catggacgagctgtacaagtaag

SEQ ID NO: 8 - DNA, 24 bp, Artificial Sequence
  Description of Artificial Sequence Synthetic oligonucleotide
cctctaactcgattgggtcaatag

SEQ ID NO: 9 - DNA, 22 bp, Artificial Sequence
  Description of Artificial Sequence Synthetic oligonucleotide
ttcgagggaacgtttatctatc

SEQ ID NO: 10 - DNA, 22 bp, Artificial Sequence
  Description of Artificial Sequence Synthetic oligonucleotide
aattacaggctacccttttgaa

SEQ ID NO: 11 - DNA, 26 bp, Artificial Sequence
  Description of Artificial Sequence Synthetic oligonucleotide
cctctccaggatcgagcatcttgaag

SEQ ID NO: 12 - DNA, 24 bp, Artificial Sequence
  Description of Artificial Sequence Synthetic oligonucleotide
tggagttggtccaacgacacctca

SEQ ID NO: 13 - DNA, 63 bp, Artificial Sequence
  Description of Artificial Sequence Synthetic oligonucleotide
  modified_base (21)..(56): a, c, t, g, unknown or other
aaaaaaagagctgacttttcnnnnnnnnnnnnnnnnnnnnnnnnnnnnnnnnnnnnatcgaat